(19) United States
(12) Patent Application Publication
(10) Pub. No.: US 2023/0183677 A1
(43) Pub. Date: Jun. 15, 2023

(54) PREDICTION, BIOSYNTHESIS, AND INTEGRATION AS BIOSENSORS OF MOLECULES WITH UNIQUE LIGHT ABSORBANCE SIGNATURES AND THEIR SUBSEQUENT IN-FIELD REMOTE DETECTION USING MULTI OR HYPER-SPECTRAL CAMERAS

(71) Applicant: Massachusetts Institute of Technology, Cambridge, MA (US)

(72) Inventors: Christopher A. Voigt, Belmont, MA (US); Yonatan Chemla, Cambridge, MA (US); Itai Levin, Cambridge, MA (US)

(73) Assignee: Massachusetts Institute of Technology, Cambridge, MA (US)

(21) Appl. No.: 17/961,925

(22) Filed: Oct. 7, 2022

Related U.S. Application Data
(60) Provisional application No. 63/288,494, filed on Dec. 10, 2021.

(51) Int. Cl. C12N 15/10

(52) U.S. Cl. CPC C12N 15/1055

(57) ABSTRACT

Disclosed herein are biosensors that are hyperspectral reporters. The biosensors are useful for detecting information about the environment and environmental conditions in a number of fields including agriculture. Also provided are methods of making and using the biosensors.

### RELATED APPLICATIONS

This application claims the benefit under 35 U.S.C. 119(e) of U.S. provisional application No. 63/288,494 filed Dec. 10, 2021 which is incorporated by reference herein in its entirety.

### GOVERNMENT FUNDING

This invention was made with government support under HQ00342010020 awarded by the U.S. Department of Defense. The government has certain rights in the invention.

### BACKGROUND OF THE INVENTION

Climate change mitigation and environmental and agricultural sustainability are important areas for development. Biosensors such as nanoparticles/nanomaterials, polymers and microbe-biosensors are now being used around the globe for solving some of the challenges in agricultural and the environment such as in sustainable food production. Biosensors can be used to monitor levels of biochemical and other categories of contaminants and pathogens that could affect agricultural produce. Biosensing has many applications, including pathogen sensors to emerging technologies in fields like defense and agriculture. A new generation of biosensors that utilize living cells instead of the classical biological-electronic hybrids biosensors based on enzymatic reactions holds promise in these areas. However, a significant limitation for the employment of living biosensors is their weak transducing capacities, which are needed to translate the signal from the sensing circuit to a machine or human-readable format.

### SUMMARY OF THE INVENTION

The present disclosure is based, at least in part, on the development of highly sensitive biosensors for use in a variety of indications including agriculture, environmental issues, diseases and other mammalian conditions, military etc. The biosensor systems disclosed herein produce signals that can be detected and processed at large distances from the biosensors, providing significant commercial advantages in many fields.

Accordingly, one aspect of the present disclosure provides a hyperspectral reporter comprising, an engineered cell comprising a transducer comprising a DNA molecule having a regulatory element responsive to a target input operatively linked to a DNA sequence encoding a signaling protein, wherein the transducer produces a detectable signal in response to the target input by producing the signaling protein, wherein the detectable signal features a unique hyperspectral signature.

In some embodiments the engineered cell is a bacterium or fungi. In some embodiments the engineered cell is a plant cell.

In some embodiments the regulatory element is an inducible promoter.

In some embodiments the signaling protein comprises the unique hyperspectral signature. In some embodiments the signaling protein is an enzyme and wherein the enzyme catalyzes the production of a metabolite comprising the unique hyperspectral signature from a substrate. In some embodiments the substrate is a naturally occurring compound in the cell. In some embodiments the signaling protein can trigger one or more steps in a biological pathway leading to the production of a compound comprising the unique hyperspectral signature.

In some embodiments the cell expresses a receptor capable of recognizing the target input. In some embodiments rein the cell comprises an exogenous nucleic acid encoding the receptor. In some embodiments the cell comprises a peptidoglycan receptor.

In some embodiments the detectable signal is produced by the unique hyperspectral signature of a compound selected from the group consisting of dihydrofolic acid, quercetin, bacteriochlorophyll a, bacteriochlorophyll b, oscillaxanthin, astaxanthin, beta-carotene, bacterioruberin, 4,4′-diapolycopenedial, protoporphyrin IX, naringenin-chalcone, and kaempferol, biliverdin-IX-δ, bisnoryangonin, and dimers of psilocin derivates. In some embodiments the detectable signal is produced by the unique hyperspectral signature of a compound selected from the group consisting of E. coli metabolites, volatile metabolites, diterpenoids, phycocyanobilin. In some embodiments the detectable signal is produced by the unique hyperspectral signature of a compound having greater than or equal to 6, 7, 8, 9, 10, 11, 12, 13, 14, 15, or 16 conjugated π-bonds. In some embodiments the detectable signal absorbs light at a wavelength of 290 nm-460 nm or 790-810 nm.

In some embodiments the target input is selected from a toxin, radiation, a pollutant, a nucleic acid, an explosive, and a nutrient. In some embodiments, the detectable signal represents a decreased level of an environmental nutrient, wherein the level is decreased with respect to a normal threshold level.

In some embodiments the cell comprises 1-50 transducers, each transducer comprising a unique DNA molecule. In some embodiments the cell comprises 1-20 transducers, each transducer comprising a unique DNA molecule. In some embodiments the cell comprises 1-12 transducers, each transducer comprising a unique DNA molecule.

In other aspects a method for detecting a target input is provided. The method involves detecting a signal having a unique hyperspectral signature, wherein the signal is generated from a hyperspectral reporter that comprises a living cell, wherein the signal provides information about the detection of the target input. In some embodiments the living cell of the hyperspectral reporter is an engineered cell comprising a transducer comprising a DNA molecule having a regulatory element responsive to the target input operatively linked to a DNA sequence encoding a signaling protein, wherein the transducer produces the detectable signal in response to the target input by producing the signaling protein.

In some embodiments the signal is detected remotely, at least about 10 meters away from the living cell. In some embodiments the signal is detected remotely, at least about 100 meters away from the living cell. In some embodiments the signal is detected using a hyperspectral camera. In some embodiments the hyperspectral camera is mounted on a drone airplane, or satellite.

In some embodiments the living cell is a bacterium or fungi. In some embodiments the signaling protein is an enzyme and wherein the enzyme catalyzes the production of a metabolite comprising the unique hyperspectral signature from a substrate. In some embodiments the substrate is a naturally occurring protein in the cell. In some embodiments the signaling protein comprises the unique hyperspectral signature. In some embodiments the signaling protein can trigger one or more steps in a biological pathway leading to the production of a compound comprising the unique hyperspectral signature.

In some embodiments the target input is selected from a toxin, radiation, a pollutant, a nucleic acid, an explosive, and a nutrient. In some embodiments the nucleic acid is a pathogen nucleic acid. In some embodiments the nucleic acid is a human nucleic acid.

In some embodiments the living cell is a plant cell. In some embodiments the target input is a nutrient and wherein the detectable signal is generated when the nutrient is present in a decreased level, wherein the level is decreased with respect to a normal threshold level. In some embodiments the target input is a nutrient and wherein the detectable signal is generated when the nutrient is present at a level within a normal range for that nutrient in that environment. In some embodiments the target input is present on crop seeds. In some embodiments the target input is derived from soil.

In some embodiments at least 2 detectable signals are generated from the living cell. In some embodiments 2-50 detectable signals are generated from the living cell. In some embodiments 2-20 detectable signals are generated from the living cell.

In other aspects a method for preparing a hyperspectral transducer, is provided. The method involves identifying a compound comprising a unique hyperspectral signature, identifying a signaling protein that can trigger one or more steps in a biological pathway leading to the production of the compound comprising the unique hyperspectral signature, and preparing a DNA molecule having a regulatory element operatively linked to a DNA sequence encoding the signaling protein. In some embodiments the method further comprises preparing a hyperspectral reporter by preparing an engineered cell transfected with the hyperspectral transducer. In some embodiments the regulatory element is a promoter responsive to the target input.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIG. 1 diagrammatically represents a living hyperspectral reporter system. Signals are received by the microbe reporter via a detectable target input (e.g. explosive tracer, radiation, pathogenic DNA, etc.) specifically activating an inducible regulatory protein. In turn, the inducible regulatory protein activates the expression of a protein-based reporter system which produces a detectible signal with hyperspectral photochemical properties.

FIGS. 2A-2B shows genetic engineering of Rubrivivax gelatinosus to function as a biosensor. FIG. 2A shows a schematic of an example genetic construct engineered in the genome of R. gelatinosus which controls production of yellow fluorescent protein (YFP). FIG. 2B shows a response curve with different concentrations of C14-homoerine lactone (HSL) determined via flow cytometry wherein FITC signal corresponds to YFP expression. The fitting curve is determined by a Hill function.

FIG. 3 shows the results of TDDFT calculations revealing 14,000 metabolites and their predicted optical absorbance properties. The radius of the plotted points is a function of the predicted intensity of the peak absorbance. The highlighted molecules correspond to promising hyperspectral reported candidates based on ease of synthesizability or predicted high-intensity absorbance near 400 nm or >650 nm. Metabolites for which no associated metabolic pathway within E. coli or B. subtilis starting points was found were labeled as “Unknown.”

FIGS. 4A-4B show Biliverdin-IX δ as a detectable hyperspectral signal. FIG. 4A shows the Biliverdin-IX δ synthesis pathway (HemQ =from B. subtilis and PigA=from P. aeruginosa). FIG. 4B shows the predicated molar absorptivity spectra of Biliverdin-IX δ.

FIGS. 5A-5B show bis-noryangonin as a detectable hyperspectral signal. FIG. 5A shows the bis-noryangonin synthesis pathway (PAL=phenylalanine ammonia-lyase; Pm4CL1=4-coumarate-CoA ligase; and PmSPS=styrylpyrone synthase). FIG. 5B shows the predicted molar absorptivity spectra of bis-noryangonin.

FIGS. 6A-6B show 2-methylpsylocin dimer as a detectable hyperspectral signal. FIG. 6A shows the structure of 2-methylpsylocin dimer. FIG. 6B shows the predicted molar absorptivity spectra of 2-methylpsylocin.

FIGS. 7A-7G shows that experimental analysis of the properties of near-UV spectral compounds dihydrofolic acid and quercetin matches their respective TDDFT-predicted photochemical properties. FIG. 7A shows the structure of dihydrofolic acid. FIG. 7B depicts the simulated molar absorptivity spectra of dihydrofolic acid determined via TDDFT. FIG. 7C depicts the experimental absorption spectra of dihydrofolic acid solutions at concentrations of 50 μM and 150 μM using Tris and Brevibacterium frigoritolerans AE31 suspended in water as controls. FIG. 7D shows the structure of quercetin. FIG. 7E depicts the quercetin biosynthesis pathway (TAL=tyrosine 4-ammonia lyase; 4CL=4-coumarate-CoA ligase; CHS=chalcone synthase; CHI=chalcone isomerase; F3H=flavanol 3β-hydroxylase; FS=flavanol synthase; FMO=flavonoid 3-monooxygenase; CPR=cytochrome P450 reductase). FIG. 7F depicts the simulated molar absorptivity spectra of intermediates in the quercetin biosynthesis pathway determined via TDDFT. FIG. 7G depicts the experimental absorption spectra of quercetin solutions at concentrations of 5 μM, 10 μM, and 15 μM and Brevibacterium frigoritolerans AE31 suspended in water as background spectra. This data reflects hyperspectral photochemical qualities of dihydrofolic acid and quercetin in the near-UV range providing unique capabilities as reporter molecules.

FIGS. 8A-8E shows remote imaging results of the near-infrared spectral reporter bacteriochlorophyll a FIG. 8A shows the structure of bacteriochlorophyll a. FIG. 8B shows the predicted molar absorptivity spectra of bacteriochlorophyll a. FIG. 8C depicts the experimental setup of plates loaded with either no hyperspectral compound, M50, or M100 densities of purified bacteriochlorophyll a. FIG. 8D shows imaging data of near-infrared bacteriochlorophyll a captured via a remote drone-mounted hyperspectral imaging device using the settings outlined in FIG. 8E. FIG. 8E shows the settings employed by the remote drone-mounted hyperspectral imaging device used to capture the image presented in FIG. 8D. This data reflects hyperspectral photochemical qualities of bacteriochlorophyll in the near-infrared range providing unique capabilities as reporter molecules.

FIGS. 9A-9B shows genetic engineering of Rubriviviax gelatinosus to function as a hyperspectral reporter. FIG. 4A shows an example of genomic cassette engineered in R. gelatinosus to control the production of bacteriochlorophyll a. FIG. 4B shows the measured hyperspectral spectrum of wildtype (WT) bacteria, the engineered bacteria, and a control bacteria.

FIGS. 10A-10B shows that the near-infrared spectral properties of bacteriochlorophyll a are detectable in complex soils. FIG. 10A shows spectral analysis of images of surface soil from the Israeli-Lebanon border displayed using imaging devices that filter either visible wavelengths (reg, green, blue (RGB)) or near-infrared wavelengths (789 nm/960 nm), or through spectral angle mapping (SAM). FIG. 10B shows spectral analysis of images of mixed sand displayed using imaging devices that either filter visible wavelengths (reg, green, blue (RGB)) or near-infrared wavelengths (789 nm/960 nm).

FIGS. 11A-11G show detection of bacteriochlorophyll a in living cells seeded on soil. FIG. 11A shows flow cytometry analysis of promoter strengths in R. gelatinosus strains that can be used for the expression of bacteriochlorophyll a. FIG. 11B shows the quantification of the flow cytometry data from FIG. 11A. FIG. 11C shows reflectance spectra of soil seeded with either no cells or R. gelatinosus expressing bacteriochlorophyll a which was captured by a remote drone-mounted hyperspectral imaging device. FIG. 11D shows images captured via a drone-mounted hyperspectral imaging device of a experiment site comprising yellow sand and black soil patches seeded with engineered R. gelatinosus. FIG. 11E shows reflectance spectra of soil seeded with varying concentrations of R. gelatinosus expressing bacteriochlorophyll a taken using a remote drone-mounted hyperspectral imaging device. FIG. 11F shows limit of detection capabilities for the analysis of soil inoculated with bacteriochlorophyll a-expressing bacteria. FIG. 11G shows computation analysis of soil samples inoculated with bacteriochlorophyll a expressing bacteria. Correcting signal in the image to select for longer wavelengths (˜800-875nm) reveals a reflectance signal highlighting the bacterial hyperspectral reporter. These results indicate that bacteria comprising a hyperspectral reporter are distinguishable from the components of complex soils.

FIGS. 12A-12I show long-term classification data of hyperspectral reporters. FIG. 12A shows an image of a experiment site taken at 120 m above ground using a filter that selects for red, green, and blue (RGB) wavelengths. The four square patches in the center of the image correspond to plots seeded with control samples or hyperspectral reporter-comprising bacteria that were inoculated at either 24, 96, 188 (control), or 189 hrs prior to imaging. FIG. 12B shows imaging analysis results of the image shown in the FIG. 12A. FIG. 12C shows automatic pixel area grouping of the plots imaged in FIG. 12A. FIG. 12D shows a histogram representing the data displayed in FIG. 12C. FIG. 12E shows quantification of a second biological replicate of the experiment described in FIG. 12A. FIG. 12F shows a histogram representing the data displayed in FIG. 12E. FIG. FIG. 12G shows data analysis to resolve a response curve and detection limit of the hyperspectral reporters. FIG. 12H shows the response curve of hyperspectral detection depending on the concentration of bacteria within a plot using bacteria that have been stored at 4° C. for one month. FIG. 121 shows the response curve of hyperspectral detection depending on the concentration of bacteria within a plot using fresh bacteria.

FIGS. 13A-13D show imaging results of an experiment site obtained using satellite imaging. FIG. 13A shows reflectance signatures of hyperspectral and multispectral reporters obtained by imaging four plots at 54 (plot 1), 125 (plot 2), 219 (plot 3), and 218 (control plot) hrs post-inoculation with bacteria. FIG. 13B shows imaging analysis results of images from a second biological replicate of the experiment described in FIG. 13A wherein the three experimental plots were loaded with bacteria 54 (plot 1), 136 (plot 2), and 218 (plot 3) hrs prior to imaging. FIG. 13C shows automatic pixel area grouping of plots from the images shown in FIG. 13B. FIG. 13D shows a histogram representing the data displayed in FIG. 13C.

### DETAILED DESCRIPTION OF THE INVENTION

Emerging technologies based on biosensors in fields such as defense and agricultural have high commercial potential. Traditional biosensors such as biological-electronic hybrid biosensors based on chemical or enzymatic reactions, i.e. glucose biosensors or pathogen biosensors, however, have limitations that minimize their efficient use in these areas. Thus, emerging technologies require a new generation of biosensors to effectively produce and capture signals in a commercially meaningful way.

A new set of sensors capable of functioning as biosensors in a wide area of technologies are disclosed herein. The new sensors utilize biological pathways in living cells to exploit signals and achieve robust, controlled output. Although living cells have been used as biosensors in some systems, a significant limitation for the employment of living biosensors is their weak transducing capacities. The ability to translate a signal produced in a living cell from a sensing circuit to a machine or human-readable format is limited in both strength and proximity. A new system of biological reporters have been designed to utilize hyperspectral imaging technologies. It has been discovered that a system based on these reporters is sufficient to enable robust and remote detection of transducer signals in living cells.

Thus, the present disclosure in some aspects involves the surprising discovery that living cells may be used as biological sensors in systems requiring strong and/or remote signals. The methods utilize hyperspectral imaging of biosensors. Hyperspectral imaging is a process which involves capturing a range of the light-reflectance spectrum (e.g. 400-1000 nm, 240-600 nm, 800-2000 nm) for each one of the pixels in an image, with high spectral resolution. This capability enables the detection of slight shifts in the light interactions of the molecules of each pixel under both lab and field conditions, making it an extremely powerful system. Hyperspectral imaging methods have been described in other contexts, for instance, in Adão, T. et al. Hyperspectral Imaging: A Review on UAV-Based Sensors, Data Processing and Applications for Agriculture and Forestry. Remote Sensing 9, 1110 (2017).

Biosensors which utilize hyperspectral imaging, as disclosed herein, involve a multi-component detection platform with photochemical properties that distinguish an active reporter within a living cell from other constituents or materials in a landscape. Without being bound by any particular belief or theory, the components of a hyperspectral reporter include, but are not limited to, a living cell comprising a sensor, a transducer within the cell, and a detectable signal produced for instance by a small molecule or a pigment molecule. The photochemical properties of the detectable signal are measured by absorbance analyses using light wavelengths that fall outside of the visible spectrum. The hyperspectral reporter system may absorb light wavelengths in the near-UV range or in the near-infrared (IR) range depending on the detectable signal.

Thus, the systems disclosed herein involves a living biosensor, referred to as a hyperspectral reporter. The biosensor may be responsive to a target input such as an environmental condition and can produce a detectable signal featuring a unique hyperspectral signature, which can be captured with the hyperspectral imaging techniques and equipment.

In some embodiments it may be possible to use at least two different reporter strains which recognize the same target molecule such that multiple different read outs are achieved to provide a potentially multifaceted signal.

A hyperspectral reporter, as used herein, is a living cell that has been engineered to function as a hyperspectral biosensor. The engineered cell comprises a sensor element and at least one transducer that responds in the cell to the target input, initiating the production of the signal. A sensor element refers to a component of the hyperspectral reporter which functions as a biorecognition element capable of detecting the presence of a target molecule in its environment. The sensor is provided by the engineered cell. The sensor may be localized at the cell surface where it can survey the local environment by making physical contact with potential target molecules. Detection of a target molecule by the sensor would, therefore, rely on the extent of chemical affinity between the sensor component and the target molecule. Examples of sensors include, but are not limited to, a receptor protein, a lipoprotein, a glycoprotein, a peptidoglycan, a glycolipid, a carbohydrate, a lipid, or a peptide.

In some embodiments sensors interact with and detect toxins such as abrin, ricin, amanitin, saxitoxin, neosaxitoxin, aflatoxins, arsenic, cadmium, mercury, lead, asbestos, dioxin and dioxin-like chemicals, bisphenol A, sulfonylurea-containing substances, acrylamide, cotinine, DEET, benzophenone-3, non-dioxin-like polychlorinated biphenyls, phthalates, perfluorochemicals, polycyclic aromatic hydrocarbons, polybrominated diphenyl ethers, polybrominated biphenyl, perchlorate, parabens, NNAL, butylated hydroxyanisole, and perfluorooctanoic acid or any other environmental chemicals or toxins determined by the Centers for Disease Control and Prevention.

In some embodiments, sensors detect toxic ammunition residues like TNT and RDX or other explosives such as nitroglycerin, acetone peroxide, cellulose nitrate, PETN, HMX, C-4, alkali metal ozonides, ammonium permanganate, ammonium chlorate, azidotetrazolates, azoclathrates, benzoyl peroxide, benzvalene, 3,5 bis(trinitromethyl)tetrazole, chlorine oxides, copper(I) acetylide, copper(II) azide, cumene hydroperoxide, CXP CycloProp(-2-)enyl Nitrate (or CPN), cyanogen azide, cyanuric triazide, diacetyl peroxide, 1-Diazidocarbamoyl-5-azidotetrazole, diazodinitrophenol, diazomethane, diethyl ether peroxide, 4-Dimethylaminophenylpentazole, disulfur dinitride, ethyl azide, explosive antimony, fluorine perchlorate, fulminic acid, halogen azides: fluorine azide, chlorine azide, bromine azide, iodine azide, hexamethylene triperoxide diamine, hydrazoic acid, hypofluorous acid, lead azide, lead styphnate, lead picrate, manganese heptoxide, mercury(II) fulminate, mercury nitride, methyl ethyl ketone peroxide, nickel hydrazine nitrate, nickel hydrazine perchlorate, nitrogen trihalides:, nitrogen trichloride, nitrogen tribromide, nitrogen triiodide, nitroglycerin, nitronium perchlorate, nitrosyl perchlorate, nitrotetrazolate-N-oxides, pentazenium hexafluoroarsenate, peroxy acids, peroxymonosulfuric acid, selenium tetraazide, silicon tetraazide, silver azide, silver acetylide, silver fulminate, silver nitride, tellurium tetraazide, tert-Butyl hydroperoxide, tetraamine copper complexes, tetraazidomethane, tetrazene explosive, tetrazoles, titanium tetraazide, triazidomethane, oxides of xenon:, xenon dioxide, xenon oxytetrafluoride, xenon tetroxide, xenon trioxide, and other explosive organic compounds contain —NO₂, −ONO₂, and —NHNO₂.

In some embodiments, sensors detect biological molecules such as a cell surface protein (e.g., a receptor protein, a lipoprotein, or a glycoprotein), a viral coat protein, a spore coat protein, a secreted protein, a peptidoglycan, a glycolipid, a carbohydrate (e.g., a membrane-inserted polysaccharide or a polysaccharide found in a cell wall of an organis,), a lipid, a specific DNA molecule, or a peptide. In some embodiments, said biological molecule is naturally found in a human pathogen for example a virus selected from the family of Adenoviridae, Picornaviridae, Herpesviridae, Hepadnaviridae, Coronaviridae, Flaviviridae, Retroviridae, Orthomyxoviridae, Paramyxoviridae, Papovaviridae, Polyomavirus, Poxviridae, Rhabdoviridae, and Togaviridae, a bacteria selected from the group of Mycobacterium tuberculosis, Streptococcus, Pseudomonas, Shigella, Campylobacter, and Salmonella, or a fungus species such as Candida, Aspergillus, Cryptococcus, Histoplasma, Pneumocytis, or Stachybotrus, Bacillus anthracis, Clostridium botulinum, Mycobacterium leprae, Yersinia pestis, Rickettsia prowazekii, Bartonella spp., or another organism such as a parasite that causes malaria, amoebiasis, babesiosis, giardiasis, toxoplasmosis, cryptosporidiosis, trichomoniasis, Chagas disease, leishmaniasis, African trypanosomiasis (sleeping sickness), Acanthamoeba keratitis, and primary amoebic meningoencephalitis (naegleriasis). In some embodiments, said biological molecule is naturally found in an agricultural pathogen for example, a fungi belonging to the phylas Ascomycota such as Fusarium spp., Thielaviopsis spp., Verticullium spp., Magnaporthe grisea, Sclerotinia sclerotiorum or Basidiomycota such as Ustilago spp., Rhizoctonia spp., Phakospora pachyrhizi, Puccinia spp., or Armillaria spp., a bacteria such as Burkholderia, Xanthomonas spp., Pseudomonas spp., Phytoplasma, Spiroplasma, other organisms such as nematodes, Phytomonas, Cephaleuros, broomrape, mistletoe, dodder, Pythium spp., Phytophthora spp., Plasmodiophora, or Spongospora. In some embodiments, said biological molecule is naturally found in a pathogen associated with crop diseases such as banana bunchy top, black bunchy top, black sigatoka, Panama disease, Fusarium head blight, powdery, mildew, barley stem rust, African cassava mosaic disease, bacteria blight, cassava brown streak disease, bacterial blight, Fusarium wilt, Verticillium wilt, Aspergillus ear rot, Giberella stalk and ear rot, grey leaf spot, basal stem rot, bud rot, groundnut rosette disease, potato brown rot, late blight, Phoma stem canker, Sclerotinia stem rot, rice clast, rice bacterial blight, sheath blight, Anthracnose, Turcicum leaf blight, soybean cyst nematode disease, Asian soybean rust, Cercospora leaf spot, rhizomania, Ratoon stuntling, red rot, sweet potato virus disease, late blight, tomato yellow leaf curl, Fusarium head blight, wheat stem rust, wheat yellow rust, anthracnose, or yam mosaic disease.

In some embodiments any of the molecules described in: Meyer, Adam J., et al. “Escherichia coli “Marionette” strains with 12 highly optimized small-molecule sensors.” Nature chemical biology 15.2 (2019): 196-204 are compounds that interact with sensors and are detectable according to the methods provided herein. In some embodiments, a molecular sensor is naturally encoded by the genome of the engineered cell and/or naturally synthesized by the engineered cell. In other embodiments, the molecular sensor is expressed off an exogenous vector (e.g. a plasmid) encoding the sensor or synthesized by proteins exogenously expressed off a vector. In other embodiments the molecular sensor may be produced by a nucleic acid integrated into the genome.

An engineered cell may be any type of cell that can be manipulated to include one or more exogenous nucleic acids. Examples of an engineered cell include, but are not limited to: bacterial cells, such as E. coli, P. putida, B. subtilis, B. frigotolerans, Pantoea agglomerans, Rubriviviax gelatinosus, Cereibacter sphaeroides, Rhodobacter capsulatus; plant cells, such as Arabidopsis, potato, tobacco and crops like wheat and corn; mammalian cells, such as human cells; fungal cells, and yeast cells, which have been genetically altered to express one or more proteins that make up aspects of the hyperspectral reporter system. Bacterial cells include but are not limited to E. coli, Rubrivivax gelatinosus. Mammalian cells include, but are not limited to Vero cells, HeLa cells, Cos cells, and CVI cells.

“Exogenous” with respect to genes indicates that the nucleic acid or gene is not in its natural (native) environment. For example, an exogenous gene can refer to a gene that is from a different species. In contrast, “endogenous” with respect to genes indicates that the gene is in its native environment. As used herein, the terms “endogenous” and “native” are used interchangeably.

The engineered cell includes one or more exogenous nucleic acids, or genetic circuits, such as a transducer. Transducer refers to the component of the hyperspectral reporter which couples the binding of the sensor component to its target to the production of the detectable signal. Thus, a transducer, as used herein, is a nucleic acid, such as a DNA molecule which encodes a signaling protein. Thus, the sensor detects the presence or absence of a compound and initiates or inhibits a signaling pathway that causes the transducer to function, leading to the production of a detectable signal (wherein the detectable signal may arise from the increase or decrease in expression of the signaling protein).

In some embodiments, proteins or other small molecules are part of the pathway and contribute to the function of transducer. In some embodiments proteins or other small molecules upstream of the transducer may bind to (alone or in combination with other compounds) and activate or inhibit expression from the transducer. In some embodiments the expressed signaling protein may interact with one or more other proteins or small molecules downstream to produce the detectable signal . In some embodiments, the sensor directly acts on the transducer without the need for any other proteins or compounds. For example, wherein a target crosses the cell membrane and binds an intracellular protein (sensor) that regulates gene expression by interacting with the transducer. Additionally, a membrane protein may be internalized upon responding to a target and in turn modulate gene expression by interacting with the transducer.

In some embodiments, the transducer comprises a nucleic acid expressing a single gene. In some embodiments, the gene encoded by the transducer comprises a sequence of a native protein or an exogenous protein in its wild-type form. In some embodiments, the gene encoded by the transducer comprises a sequence of a protein having one or more mutations (e.g., substitutions, deletions, or additions) relative to a wild-type or native form of the protein. In some embodiments, the gene encoded by the transducer may comprise a truncated version of protein such as a protein comprising select functional domains that can contribute to the transduction process. In some embodiments, the gene encoded by the transducer may comprise a fusion protein of one or more polypeptides optionally connected via a linker sequence.

In some embodiments, the transducer may comprise one or more nucleic acids encoding multiple genes. For example, in some embodiments, the transducer may comprise multiple genes provided on the same nucleic acid molecule or on different nucleic acid molecules. In some embodiments, the transducer may comprise a single gene or set of genes provided from an exogenous nucleic acid (such as a vector) and a single gene or set of genes provided from a site within the genome of a bacteria comprising the exogenous nucleic acid.

In some embodiments, transducers comprising multiple genes may be provided in an operon. In some embodiments, the transducer may be codon optimized. In some embodiments, the nucleic acid has a regulatory element responsive to a target input operatively linked to a DNA sequence encoding a signaling protein. The nucleic acid, thus, in some embodiments, comprises a DNA molecule having a regulatory element operatively linked to a DNA sequence encoding the signaling protein. As used herein, “a regulatory element” is the DNA sequence which is necessary to initiate or disrupt the transcription of the gene. Operatively linked means that the regulatory element is connected to the nucleic acid encoding the protein in a manner that it can direct the transcription of the linked protein-coding DNA sequence based on interaction with other components in the local environment of the nucleic acid. In some embodiments, the transducer, or at least one gene of a multi-gene transducer, are provided on an exogenous vector. In some embodiments the transducer may be produced by a nucleic acid integrated into the genome.

Transducers fulfill signaling roles within the engineered cell hyperspectral reporter system. Examples of transducers include, but are not limited to, nucleic acids expressing signaling proteins that are naturally expressed by the engineered cell or exogenously expressed by the engineered cell of a vector. Signaling proteins useful herein, in some embodiments, are proteins that modulate the activity of another protein within a cell or catalyze a metabolic process in a cell. Examples of said transducers include, but are not limited to, kinases, phosphatases, lipid-transferases, glycosylases, ubiquitin ligases, methyltransferases, acetyltransferases, SUMO transferases, proteases, allosteric modulators, foldases, and metabolic enzymes (e.g., lyases, dehydrogenases, phosphorylates, dephosphorylases, kinases, transferases, synthases, hydrolases, etc.). In some embodiments, the signaling protein encoded by the nucleic acid transducer is a protein capable of generating a signal featuring a unique hyperspectral reporter. In some embodiments, the signaling protein encoded by the nucleic acid transducer is a protein involved in a metabolic pathway that generates a signal featuring a unique hyperspectral reporter. In some embodiments, a transcription factor or transcriptional repressor can contribute to the transducer activity. In some embodiments, the signaling protein is a translational regulator such that it can regulate protein synthesis by affecting ribosome activity.

In some embodiments, the transducer is purified from cells prior to exposing it to a target.

Transducers may be made up of one individual protein capable of providing communication between the sensor and the machinery that produces the detectable signal. Alternatively, transducers may be made up of multiple proteins, thereby comprising a multi-step signaling network, that is responsible for relaying communication between the sensor and the machinery that produces the detectable signal. Examples of transducers comprising a multi-step signaling network include, but are not limited to, enzymes that synthesize quercetin from L-tyrosine which are 4-ammonia lyase, 4-coumarate-CoA ligase, chalcone synthase, chalcone isomerase, 3β-hydroxylase, flavanol synthase, flavonoid 3-monooxygenase, and cytochrome P450 reductase, enzymes that synthesize bacteriochlorophyll a from protoporphyrin IX which are the magnesium chelatase enzyme complex, MgP methyltransferase, MgP monomethyl ester cyclase, bacteriochlorophyll a hydratase, bacteriochlorophyll a dehydrogenase, bacteriochlorophyll a reductase, and bacteriochlorophyll a synthase, enzymes that synthesize biliverdin-IX δ from Fe-coproporphyrin III which are HemQ and PigA, enzymes that synthesize 2-methylpsylocin such as L-Tryptophan 2-C-methyltransferase, tryptophan decarboxylase, tryptamine 4-monooxygenase, tryptamine N-methyltransferase, and Laccase or cytochrome P450, and enzymes that synthesize bis-noryangonin from tyrosine which are phenylalanine ammonia-lyase, 4-coumarate-coenzyme A ligase, and styrylpyrone synthase.

In some embodiments a reporter cell can be used to detect multiple triggers. For instance, in some embodiments a first target may trigger the production of a sensor for a second target. Thus, a reporter cell can conditionally report on the presence of a second molecule only when a first molecule is present, thus increasing the complexity of the detection process.

In some embodiments, the nucleic acid has a regulatory element responsive to a target input operatively linked to a DNA sequence encoding a signaling protein. The nucleic acid, thus, in some embodiments, comprises a DNA molecule having a regulatory element operatively linked to a DNA sequence encoding the signaling protein. As used herein, “a regulatory element” is the DNA sequence which is necessary to initiate or disrupt the transcription of the gene. Operatively linked means that the regulatory element is connected to the nucleic acid encoding the protein in a manner that it can direct the transcription of the linked protein-coding DNA sequence based on interaction with other components in the local environment of the nucleic acid. In some embodiments, the regulatory element is a promoter that may be activated by a cellular signal, such as the presence of a target input. In other embodiments, the regulatory element may be an enhancer or repressor, ribosome binding sites, ribozymes, enhancer sequences, response elements, protein recognitions, inducible elements, protein binding sites, 5′ and 3′ untranslated regions (UTRs), transcriptional start sites, transcription terminator sequences, or combinations thereof.

A regulatory element that is responsive to a target input, as used herein, is an element that either increases or decreases the expression of the signaling protein following a change in the cellular environment as a result of exposure or lack thereof to a target input. The exposure may be direct or indirect. For instance, the exposure may be direct if the target input enters the cell and binds directly to the regulatory element, influencing the activity of that regulatory element. More commonly the exposure is indirect and the target input acts or fails to act on a separate component of the cells, such as a receptor, which initiates a signal or signaling pathway which ultimately interacts with or fails to interact with the regulatory element. A target may negatively regulate the expression through the regulatory element by disrupting a positive regulator or promoting a negative regulator.

Thus, the regulatory elements may also be activation elements or inhibitory elements. An activation element is a nucleic acid sequence that when presented in context with a nucleic acid to be expressed, will cause expression of the nucleic acid in the presence of an activation signal. An inhibitory signal is a nucleic acid sequence that when presented in context with a nucleic acid to be expressed will cause expression of the nucleic acid unless an inhibitory signal is present. Each of the activation and inhibitory elements may be a promoter, such as a bacteriophage T7 promoter, sigma 70 promoter, sigma 54 promoter, lac promoter, etc. As used herein, the term “promoter” is intended to refer to those regulatory sequences which are sufficient to enable the transcription of an operably linked DNA molecule. Promoters may be constitutive or inducible. As used herein, the term “constitutive promoter” refers to a promoter that is always on (i.e. causing transcription at a constant level). Examples of constitutive promoters include, without limitation, sigma 70 promoter, bla promoter, lacI. promoter, etc. Non-limiting examples of inducible promoters include ParaBAD, PrhaBAD, Plac, Ptac, Plux, Ptet, Psa1, Ptrp, PA1lacO1 and Ppho.

Thus, in some embodiments, the transducer is constitutively expressed or in a continuously active state whereby the transducer is constantly promoting the production of the detectable signal such that the target input inhibits the activity of the transducer. In some embodiments, the transducer is in an inactive state such that the target input inhibits the activity of the transducer. Transducers may be activated or inhibited through a variety of mechanisms. In some embodiments, the target input directly activates or inhibits the transducer (e.g., a small molecule interacting with the nucleic acid encoding the transducer). In some embodiments, the target input activates a factor which regulates the expression of the transducer (e.g., triggering a signaling cascade that effects transcription of the transducer or allosterically modulating a transcriptional regulator of the transducer).

Inducible promoters allow regulation of gene expression and can be regulated by target inputs, i.e. environmental factors such as temperature, the presence of exogenous chemicals, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only.

As used herein, the term “terminator” (as referred to as a transcription terminator) is a section of nucleic acid sequence that marks the end of a gene or operon in genomic DNA during transcription. Terminators stop transcription of a polymerase. Terminators can be classified into several groups. At the first group of termination signals the core enzyme can terminate in vitro at certain sites in the absence of any other factors (as tested in vitro). These sites of termination are called intrinsic terminators or also class I terminators. Intrinsic terminators usually share one common structural feature, such as a hairpin or stem-loop structure. On the one hand the hairpin comprises a stem structure, encoded by a dG-dC rich sequence of dyad symmetrical structure. On the other hand the terminator also exhibits a dA-dT rich region at the 3′-end directly following the stem structure. The uridine rich region at the 3′ end is thought to facilitate transcript release when RNA polymerase pauses at hairpin structures. Two or more terminators can be operatively linked if they are positioned to each other to provide concerted termination of a preceding coding sequence. The terminator sequences may be downstream of coding sequences, i.e. on the 3′ position of the coding sequence. The terminator can e.g. be at least 1, at least 10, at least 30, at least 50, at least 100, at least 150, at least 200, at least 250, at least 300, at least 400, at least 500 nucleotides downstream of the coding sequence or directly adjacent. Examples of terminators include, but are not limited to, T7 terminator, rrnBT1, L3S2P21, tonB , rrnA , rrnB , rrnD, RNAI, crp, his, ilv lambda, M13, rpoC, and trp.

The transducer is able to signal in response to the target input by producing the signaling protein. The signaling protein, as used herein is a protein that either itself features a unique hyperspectral signature or initiates a reaction in the cell, or a series of reactions (e.g., a metabolic pathway) that results in the production of the compound having a unique hyperspectral signature, which serves as a detectable signal. The signaling protein may be, for instance, an enzyme that catalyzes the production of a compound having a unique hyperspectral signature from a substrate. Alternatively, the signaling protein may initiate a pathway leading to the production or activation of a compound having a unique hyperspectral signature. Each of these components, other than the signaling protein, may be native to the cell. For instance, the nucleic acid encoding the signaling protein may be the only exogenous molecule in the cell. In some other embodiments one or more other proteins in a signaling pathway may be expressed from an exogenous nucleic acid in the cell. In some embodiments, the signaling proteins or the set of signaling proteins are endogenous within the engineered cell.

In some embodiments the signaling protein is an enzyme and wherein the enzyme catalyzes the production of a metabolite comprising the unique hyperspectral signature from a substrate. In some embodiments, the signaling protein is a regulator of metabolic enzyme that can influence the activity of a metabolic pathway in the presence or absence of a target input. Any enzyme that can directly or indirectly produce the compound having a unique hyperspectral signature is useful in the methods disclosed herein. Non-limiting exemplary enzymes useful in these methods include 4-ammonia lyase, 4-coumarate-CoA ligase, chalcone synthase, chalcone isomerase, 3β-hydroxylase, flavanol synthase, flavonoid 3-monooxygenase, cytochrome P450 reductase, magnesium chelatase enzyme complex, MgP methyltransferase, MgP monomethyl ester cyclase, bacteriochlorophyll a hydratase, bacteriochlorophyll a dehydrogenase, bacteriochlorophyll a reductase, bacteriochlorophyll a synthase, HemQ, PigA, L-Tryptophan 2-C-methyltransferase, tryptophan decarboxylase, tryptamine 4-monooxygenase, tryptamine N-methyltransferase, Laccase, cytochrome P450, phenylalanine ammonia-lyase, 4-comurate-coenzyme A ligase, and styrylpyrrone synthase. Non-limiting exemplary substrates that may be used by signaling proteins of the present disclosure include Fe-copoporphyrin III, protoheme, L-tyrosine, p-coumaric acid, p-coumaroyl-CoA, naringenin chalcone, naringenin, kaempferol, protoporphyrin IX, Mg-protoporphyrin IX monomethyl ester, 3,8-divinyl protochlorophyllide a, 3,8-divinyl chlorophyllide a, 3-divinyl-3-(10hydroxyethyl)chlorophyllide a, 3-acetyl-3-devinylchlorophyllide a, and bacteriochlorophyllide a.

The engineered cell may include a single transducer, or nucleic acid encoding a signaling protein. Alternatively, the engineered cell may include more than one transducer. The multiple transducers may be included within the same or within different nucleic acid vectors within the cell or within the same or different nucleic acids integrated into the genome. In some embodiments the cell comprises 1-50 transducers, 1-45 transducers, 1-40 transducers, 1-35 transducers, 1-30 transducers, 1-29 transducers, 1-28 transducers, 1-27 transducers, 1-26 transducers, 1-25 transducers 1-24 transducers, 1-23 transducers, 1-22 transducers, 1-21 transducers 1-20 transducers, 1-19 transducers, 1-18 transducers, 1-17 transducers, 1-16 transducers, 1-15 transducers 1-14 transducers, 1-13 transducers, 1-12 transducers, 1-11 transducers 1-10 transducers, 1-9 transducers, 1-8 transducers, 1-7 transducers, 1-6 transducers, 1-5 transducers 1-4 transducers, 1-3 transducers, or 2 transducers, each transducer comprising a unique DNA molecule.

In some embodiments the materials and methods disclosed herein comprise a circuit that operates on a feed-forward system. For instance, an engineered cell produces a signal in response to a target input and the target input also triggers a down stream event that produces a physiological response. In some embodiments that event may be the expression of one or more proteins, such as enzymes that produce the physiological response. For instance, the expressed enzymes may regulate a second pathway that results in the production of a biocontrol agent. In some embodiments the one or more proteins may be additional substrates and/or enzymes involved in further production of the detectable signal or another physiological response, such as a response to the initial target input, i.e., to reduce the levels of the initial target input. In some embodiments, reducing the initial target input may be achieved by reducing the responsiveness of the reporter (e.g., down-regulation of a receptor for the target input) or triggering a process that inhibits the target input (e.g., secretion of degradative enzymes or release of a biocontrol agent that kills an organism responsible for the production of the target input). Thus, in some embodiments, production of a biocontrol agent that inhibits the target input may result in the eventual decrease of the detectable signal relaying information that the environment has been rid of a pest or pathogen. In some embodiments, response of the reporter to a target input may result in the production of enzymes and/or substrates that participate in the pathway leading to the production of the detectable signal thereby amplifying the signal. In some embodiments, the response of the reporter to a target input may result in the production of proteins that are able to couple the presence of a second target input, which is distinct from the first target input, to the production of a second detectable signal featuring a unique hyperspectral signature. In such examples, the hyperspectral reporter can conditionally sense a second target input only in the presence of a first target input and therein produces two distinct detectable signal corresponding to the presence of their respective target inputs. In some embodiments, the physiological response may be the activation of a metabolic pathway that produces a nutrient in order to respond to low nutrient levels in the environment. In some embodiments, the physiological response may result in the production of a nutrient such as a vitamin. In some embodiments, the physiological response may result in the activation of a nitrogen fixation pathway. In some embodiments, the physiological response may result in the release of substances (e.g., chemical or enzymes) that increase the pH or decrease the pH of the surrounding environment, As such, the reporter may be capable of turning on a physiological response in the presence of a target input that can compensate for an imbalance in the nutrient composition or the pH of soil where a plant is growing. Or, the reporter may be capable of altering the metabolic state of a plant or seed in the soil where the reporter is deposited by producing one or more nutrients and providing it to the plant.

The detectable signal features a unique hyperspectral signature. A unique hyperspectral signature, as used herein, refers to a signature generated from a compound and that is unique to that compound, such that the hyperspectral signal that is detected from that compound positively identifies the presence of that compound in a cell or environment. The absence of such a signal representative of the unique hyperspectral signature indicates the absence of that compound in a cell or environment.

A compound having a unique hyperspectral signature is not limited by any specific example disclosed herein. There are numerous compounds that may be used in the methods, including, for instance, many naturally occurring compounds found in the engineered cells and/or the local environment of the engineered cells when the signal is detected. In some embodiments, the detectable signal is produced within a cell and does not undergo secretion. In some embodiments, the detectable signal is produced within a cell and then secreted extracellularly. In some embodiments the compound having the unique hyperspectral signature is a compound such as, for instance, a bacterial metabolite, such as an E. coli metabolite, a volatile metabolite, a diterpenoids, a phycocyanobilin-like metabolite or other biogenic compounds. A bacterial metabolite is a compound that is produced or activated within a bacteria and has a unique hyperspectral signature. In some embodiments the bacterial metabolites have greater than or equal to 6, 7, 8, 9, 10, 11, 12, 13, 14, 15, or 16 conjugated π-bonds. A volatile metabolite is a metabolite that is produced or activated within a cell and includes those described for instance in Hou et al., Biofilms Microbiomes, 7, 2 (2021). Diterpenoids are compounds that typically are derived from a substrate: (E,E,E)-geranylgeranyl diphosphate by a diterpene synthase (DTS). In some embodiments the diterpenoids are included within the Metacyc database and optionally have greater than or equal to 3 conjugated double bonds. Phycocyanobilin-like metabolites are cellular metabolites that are structurally similar to phycocyanobilin. These compounds include, for example, compounds found within the metacyc database and having a Tanimoto similarity score of great than 0.65 to phycocyanobilin. These compounds absorb in the long wavelength end of the spectrum. Many other biogenic compounds may also be used. For instance, biogenic molecules having greater than or equal to 6, 7, 8, 9, 10, 11, 12, 13, 14, 15, or 16 conjugated π-bonds. (for example to capture compounds that absorb in the UV visible range) or other important properties may be identified from the Zinc15 database, the COCONUT natural products database, or other databases. The Zinc15 database contains over 230 million compounds in in ready to dock 3D formats.

Non-limiting examples of these compounds include, but are not limited to, dihydrofolic acid, quercetin, chlorophyll a, bacteriochlorophyll a, bacteriochlorophyll b, oscillaxanthin, astaxanthin, beta-carotene, protoporphyrin IX, porphyrin, piperine, NADH, oscillaxanthin, astaxanthin, beta-carotene, melanin, biliverdin-IX δ, bis-noryangonin, 2-methylpsylocin dimer, 5,10-methenyltetrahydrofolate, coproporphyrin III, bacterioruberin, emodin dianthrone, or 4,4′-diapolycopenedial.

In some embodiments the detectable signal absorbs light at a wavelength of 290 nm-460 nm or 790-810 nm. In some embodiments, the detectable signal absorbs light at a wavelength within wavelength ranges 290 nm-300 nm, 300 nm-310 nm, 310 nm-320 nm, 320 nm-330 nm, 330 nm-340 nm, 340 nm-350 nm, 350 nm-360 nm, 360 nm-370 nm, 370 nm-380 nm, 380 nm-390 nm, 390 nm-400 nm, 400 nm-410 nm, 410 nm-420 nm, 420 nm-430 nm, 430 nm-440 nm, 440 nm-450 nm, 450 nm-460 nm, 790 nm-800 nm, or 800 nm-810 nm. In some embodiments, the detectable signal absorbs light within a wavelength range selected from the group consisting of 290 nm-300 nm, 300 nm-310 nm, 310 nm-320 nm, 320 nm-330 nm, 330 nm-340 nm, 340 nm-350 nm, 350 nm-360 nm, 360 nm-370 nm, 370 nm-380 nm, 380 nm-390 nm, 390 nm-400 nm, 400 nm-410 nm, 410 nm-42 0nm, 420 nm-430 nm, 430 nm-440 nm, 440nm-450 nm, 450 nm-460 nm, 790 nm-800 nm, and 800 nm-810 nm. In some embodiments, the detectable signal features a multispectral signature wherein the compound absorbs light at more than one (e.g. one, two , or three) wavelengths within wavelength ranges 290 nm-460 nm or 790-810 nm such as ranges selected from the group consisting of 290 nm-300 nm, 300 nm-310 nm, 310 nm-320 nm, 320 nm-330 nm, 330 nm-340 nm, 340 nm-350 nm, 350 nm-360 nm, 360 nm-370 nm, 370 nm-380 nm, 380 nm-390 nm, 390 nm-400 nm, 400 nm-410 nm, 410 nm-420 nm, 420 nm-430 nm, 430 nm-440 nm, 440 nm-450 nm, 450 nm-460 nm, 790 nm-800 nm, and 800 nm-810 nm. In some embodiments the detectable signal features a multispectral signature wherein the compound absorbs light within more than one (e.g., one, two, or three) wavelength ranges selected from the group consisting of 290 nm-300 nm, 300 nm-310 nm, 310 nm-320 nm, 320 nm-330 nm, 330 nm-340 nm, 340 nm-350 nm, 350 nm-360 nm, 360 nm-370 nm, 370 nm-380 nm, 380 nm-390 nm, 390 nm-40 0nm, 400 nm-410 nm, 410 nm-420 nm, 420 nm-430 nm, 430 nm-440 nm, 440 nm-450 nm, 450 nm-460 nm, 790 nm-800 nm, and 800 nm-810 nm.

In some embodiments, the hyperspectral reporter cells may comprise the components necessary for being able to detect target inputs and produce detectable signals related to the presence of more than one target input in an environment. For example, in some embodiments, the hyperspectral reporters may comprise two sensors capable of recognizing different, respective target inputs. In such example, the two separate sensors may communicate with the nucleic acid encoding the signaling protein by activating separate, respective transducers thereby resulting in the production of different signaling proteins capable of initiating a process resulting in the production of two separate detectable signals. Said example should not be considered limiting as hyperspectral reporters of the present disclosure may comprise engineered cells capable of sensing one, two, three, four, five, six, seven, eight, nine, ten or more target inputs.

Numerous genetic circuits to link these biosynthetic pathways to sensors, based on the guidance and teachings provided herein may be developed. Different unique hyperspectral signatures may be identified and utilized to optimize for sensitivity and specificity in the detection of spectral shifts. In some embodiments, the biosynthetic pathways involved in the development of the metabolites may be selected based on their ability to be genetically engineerable and to survive in harsh environments. For example, a purple soil bacteria named Rubrivivax gelatinosus can produce detectable levels of bacteriochlorophyll a and may be particularly useful in a variety of settings as a hyperspectral reporter system.

The systems disclosed herein respond to a target input. “Target input” refers to any stimulation of the hyperspectral reporter that leads to synthesis of the signaling protein. For instance, a target input may be a chemical structure that bears binding affinity to the sensor element and/or leads to activation (directly or indirectly) of the sensor element of the hyperspectral reporter. In some embodiments, the loss of a target input may lead to activation (directly or indirectly) of the sensor element of the hyperspectral reporter. Target inputs may be synthetic, non-biological molecules including, but not limited to, human-made explosives, human-made drug molecules, human-made polymer components of materials, toxins, radiation, or pollutants. Target inputs may also be biological molecules including, but not limited to, nucleic acids, lipids, carbohydrates, and proteins. In some embodiments the target input is a nucleic acid, an explosive, and/or a nutrient. In some embodiments, the target input is a pathogen such as a bacteria, fungus, or virus that is infectious to humans.

Detection of the signal having a unique hyperspectral signature in response to a target input may provide a variety of information about the environment. For instance, the signal may simply reflect the presence of the hyperspectral reporter in the environment. The reporter may emit a constant signal that can be detected simply to determine whether the reporter is present. This may be desirable, for instance, as a control to confirm that the absence of different signal from the reporter is in fact due to a target input rather than the absence of the hyperspectral reporter from the scanned environment. In such a case the reporter would include at least two transducers, one for the control and the other to detect the target input.

In other embodiments the detectable signal may reflect the presence of a target input in the environment, wherein the target input interacts with and activates the hyperspectral reporter. In other instances, the detectable signal may reflect the loss of a target input. For instance, presence of the target input may produce a constant signal in the hyperspectral reporter. The loss of the target input in that environment may lead to the loss of signal, which can provide information about the environment and that particular input. In other embodiments the presence of a target input may activate repression (directly or indirectly) of the sensor element of the hyperspectral reporter and thus lead to a loss of signal. The presence of the target input in this circumstance would be associated with a loss of signal.

The target input may be, for instance, a condition rather than a chemical entity or molecule. Conditions such as temperature, pH, radiation, moisture levels, etc, may serve as target inputs that stimulate the hyperspectral reporter. In such embodiments, the sensor comprises a polypeptide that responds to changes in temperature, pH, radiation, or moisture levels. The ability to detect such changes has important implications in military, energy management and agriculture.

The target input may also be a chemical entity or chemical signal. As used herein, the term “chemical signals” refers to chemical compounds. Any substance consisting of two or more different types of atoms (chemical elements) in a fixed stoichiometric proportion can be termed a chemical compound. Chemical signals can be synthetic or natural chemical compounds. In some embodiments, the chemical signal is a signal that is non-natural to the environment (e.g., toxins, explosives, metal). In some embodiments, the signal is a native biological signal (e.g. root exudate, biological control agent, etc.). Non-limiting examples of chemical signals include nutrients, viral or other pathogen particles or nucleic acids, toxins, explosives, root exudates, biocontrol agents, phytohormones, vanillate, IPTG, aTc, cuminic acid, DAPG, and salicylic acid, 3,4-dihydroxybenzoic acid, 3OC6HSL and 3OC14HSL.

In some instances, the engineered cell is a plant cell and the methods disclosed herein are useful in agriculture. For instance, it may be desirable to engineer a plant cell such that it includes a transducer that is able to generate a hyperspectral signal in response to changing environmental conditions. Such engineered plant cells can be monitored for the presence of hazardous chemicals, pathogens or conditions, or checked to determine that adequate nutrients are present. Thus, in some aspect's plants comprising one or more engineered cells are provided herein. Similar monitoring can be performed on non-modified plants using other cells such as bacteria which are added to the environment of the plant.

In some embodiments the target input is a nutrient for a plant. Examples of nutrients include macromolecules or vitamins. A detectable signal may be generated when the nutrient is present in a decreased level. For instance, when environmental levels of a particular nutrient decrease with respect to a normal threshold level of that nutrient, a detectable signal may be produced (or lost) in order to provide an alert that nutrient levels are inadequate. Alternatively, the target input may be a nutrient that produces a detectable signal when the nutrient is present at a level within a normal range for that nutrient in that environment.

The target input may be located in any part of the environment of a plant. For instance, it may be present on crop seeds, in soil, in root exudate, on or in the plant. As used herein, the term “root exudate” refers to chemicals secreted or emitted by plant roots in response to their environment. These allow plants to manipulate or alter their immediate environment. Root exudates are a complex mixture of soluble organic substances, which may contain sugars, amino acids, organic acids, enzymes, and other substances. Root exudates include, but are not limited to, ions, carbon-based compounds, amino acids, sterols, sugars, hormones (phytohormones), flavonoids, antimicrobials, and many other chemical compounds. The exudates can serve as either positive regulators or negative regulators of the hyperspectral reporters, in order to provide information about the plant or environment.

Biological control or biocontrol is a method of controlling pests such as insects, mites, weeds and plant diseases using other organisms. Natural enemies of insect pests, also known as biological control agents, include predators, parasitoids, pathogens, and competitors. Biological control agents of plant diseases are most often referred to as antagonists. Biological control agents of weeds include seed predators, herbivores and plant pathogens. The hyperspectral reporters disclosed herein may be useful for detecting changes in factors used to control pests or disease as well as the presence of such pests or diseases. For instance, modulation of the reporter by a secreted factor (or chemical otherwise associated with) a biological control agent may be used to produce a detectable signal. Herein, that is referred to as a “biocontrol agent”. Additionally, the reporters may be used to detect chemicals associated with a pest or disease (i.e., DNA) or secreted by a pest or diseased cell. In some embodiments the reporter cells may be used as a way of determining whether or not to treat an area with a biocontrol chemical and/or stop treating an area with a biocontrol chemical. In some embodiments the reporter may be able to release a biocontrol agent (e.g., a pesticide synthesized in the cell or a bacteriophage), thus serving as both a reporter for the presence of a pest/plant pathogen in addition to being a control agent.

The detection of the hyperspectral reporter is carried out by remote methods which make use of specialized cameras that sense light wavelengths that fall outside the visible spectrum. The specialized camera collects data from a hyperspectral cube in which two dimensions represent the spatial extent of the scene and the third dimension represents the spectral content of the scene. Examples of remote detection methods include, but are not limited to, hand-held devices, unmanned aerial vehicles (UAVs), and satellites.

One advantage of the methods disclosed herein is that a signal can be generated and detected remotely. A remotely detected signal refers to detection of a signal at least 5 meters away from the living cell. In some embodiments a remotely detected signal refers to detection of a signal at least 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900, or 1,000 meters away from the living cell. In some embodiments each of these distances may have an upper limit of detection of 20,000, 15,0000, 10,000, 5,000, or 1,000 meters from the living cell. The signal detection can take place, for instance, using a hyperspectral camera that is mounted on a drone or airplane.

Methods for identifying compounds with unique hyperspectral signatures have been developed. In some embodiments the methods involve a computational analysis to predict the UV-Visible absorbance spectra of organic molecules, for instance, using Time-Dependent Density Functional Theory (TD-DFT) (Jacquemin, D., Perpete, E. A., Ciofini, I. & Adamo, C. Accurate simulation of optical properties in dyes. Accounts of chemical Research 42, 326-334 (2009).). This computation analysis has been used to predict the spectra for over 15,000 metabolites. Spectra from the UV/Vis+³ experimental spectrum database were standardized, providing 4,100 absorbance spectra that can be used to benchmark and compare the optical prediction methods. Additional spectral predictions are being generated to further identify metabolite spectra.

Metabolites identified using these and other methods may be biosynthesized in living cells and used in the methods disclosed herein. For instance, two compounds, quercetin and bacteriochlorophyll a, the former in the ultraviolet and the latter in the near-infrared regions were tested as described further in the Examples. Using a Headwall E-series hyperspectral camera setup, these compounds were detected on sand and complex soil backgrounds. The detection limit was significantly better when using HSI than regular RGB imaging. Quercetin and bacteriochlorophyll were detectable at concentrations as low as 20 μM and 2 μM, respectively, when dripped on the sand, while at those concentrations, they were invisible to the naked eye or RGB detectors.

The compound detection disclosed herein was confirmed under field conditions using a UAV carrying Headwall Nano hyperspectral camera. It was possible to detect the molecules at 15 m altitude on sand. These living cells, sprayed on soil (OD_{600 nm}=2), were detected under field conditions using the UAV detection system from 15 m, both on sand and on light-absorbing black soil. Calculating the ratio between the reflectance measured at two molecule-specific wavelengths at each pixel was shown to be a simple and robust method to distinguish between signal and background.

In some embodiments of the present disclosure an exogenous nucleic acid is included in a cell, for instance it is integrated into the genome or included within a vector. The nucleic acid may be a genetic circuit, vector or expression cassette which encodes one or more proteins. Methods to introduce exogenous genetic material into a cell are well-known in the art. For example, exogenous DNA material may be introduced into the cell by calcium phosphate precipitation technology. Other technologies, such as the retroviral vector technology, electroporation, lipofection and other viral vector systems such as adeno-associated virus system, or microinjection may be used.

Regulatory elements include promoter sequences to bind RNA polymerase and translation initiation sequences for ribosome binding. For example, a bacterial expression vector may include a promoter such as the lac promoter and for translation initiation the Shine-Dalgarno sequence and the start codon ATG. Similarly, a eukaryotic expression vector may include a heterologous or homologous promoter for RNA polymerase II, a downstream polyadenylation signal, the start codon ATG, and a termination codon for detachment of the ribosome. Such vectors may be obtained commercially or assembled from the sequences described by methods well-known in the art.

To maximize the expression of the signaling protein, the sequence flanking the translation initiation codon may be modified. The nucleic acids disclosed herein may include the incorporation of codons “preferred” for expression by selected mammalian or non-mammalian hosts (i.e., codon optimized); the provision of sites for cleavage by restriction endonuclease enzymes; and the provision of additional initial, terminal or intermediate DNA sequences that facilitate construction of expression vectors. In addition, artificial introns may be introduced so as to increase the production of the protein. Other special targeting sequences such as the nuclear localization signal (such as the SV40 nuclear localization signal) may be added.

Nucleic acids, may include, in some embodiments, a nucleotide sequence encoding a protein that is at least about 85% or more homologous or identical to the entire length of a naturally occurring nucleic acid sequence, e.g., at least 5%, 10%, 15%, 20%, 25%, 30%, 35%, 40%, 50% or more of the full length naturally occurring nucleic acid sequence). In some embodiments, the nucleotide sequence is at least about 90%, 91%, 92%, 93%, 94%, 95%, 96%, 97%, 98%, 99% or 100% homologous or identical to a naturally occurring nucleic acid sequence. In some embodiments, the nucleotide sequence is at least about 85%, e.g., is at least about 90%, 91%, 92%, 93%, 94%, 95%, 96%, 97%, 98%, 99% or 100% homologous or identical to a nucleic acid sequence, in a fragment thereof or a region that is much more conserved, such as an essential, but has lower sequence identity outside that region.

Calculations of homology or sequence identity between sequences (the terms are used interchangeably herein) are performed as follows. To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

In some embodiments the nucleic acids encoding the proteins may have native gene sequences. In some embodiments, the nucleic acids are modified with respect to a native sequence. In some instances, the nucleic acids may include non-naturally occurring nucleotides and/or substitutions, i.e. Sugar or base substitutions or modifications.

One or more substituted sugar moieties include, e.g., one of the following at the 2′ position: OH, SH, SCH3, F, OCN, OCH3OCH3, OCH3O(CH2)n CH3, O(CH2)n NH2 or O(CH2)n CH3 where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of a nucleic acid; or a group for improving the pharmacodynamic properties of a nucleic acid and other substituents having similar properties. Similar modifications may also be made at other positions on the nucleic acid, particularly the 3′ position of the sugar on the 3′ terminal nucleotide and the 5′ position of 5′ terminal nucleotide. Nucleic acids may also have sugar mimetics such as cyclobutyls in place of the pentofuranosyl group.

Nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-' 7′ 7; and Gebeyehu, G., et al. Nucl. Acids Res., 15:4513 (1987)). A “universal” base known in the art, e.g., inosine, can also be included.

As used herein, the equivalent terms “expression” or “gene expression” are intended to refer to the transcription of a DNA molecule into RNA, and the translation of such RNA into a polypeptide.

As used herein, a “gene” refers to a nucleic acid sequence that encode a protein or gene product. As used herein, a “gene cluster” refers to a set of two or more genes that encode gene products.

As used herein, the term “delete” or “deleted” refers to the removal of a gene (e.g. endogenous gene) from a sequence or cluster. As used herein, the term “alter” or “altered” refers to the modification of one or more nucleotides in a gene or the deletion of one or more base pairs in a gene. This alteration may render the gene dysfunctional. Method of deletion and alteration, in the context of genes, are known in the art.

Methods to deliver expression vectors or expression constructs into cells, for example, into bacteria, yeast, or plant cells, are well known to those of skill in the art. Nucleic acids, including expression vectors, can be delivered to prokaryotic and eukaryotic cells by various methods well known to those of skill in the relevant biological arts. Methods for the delivery of nucleic acids to a cell, include, but are not limited to, different chemical, electrochemical and biological approaches, for example, heat shock transformation, electroporation, transfection, for example liposome-mediated transfection, DEAE-Dextran-mediated transfection or calcium phosphate transfection. In some embodiments, a nucleic acid construct, for example an expression construct comprising a fusion protein nucleic acid sequence, is introduced into the host cell using a vehicle, or vector, for transferring genetic material. Vectors for transferring genetic material to cells are well known to those of skill in the art and include, for example, plasmids, artificial chromosomes, and viral vectors. Methods for the construction of nucleic acid constructs, including expression constructs comprising constitutive or inducible heterologous promoters, knockout and knockdown constructs, as well as methods and vectors for the delivery of a nucleic acid or nucleic acid construct to a cell are well known to those of skill in the art.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

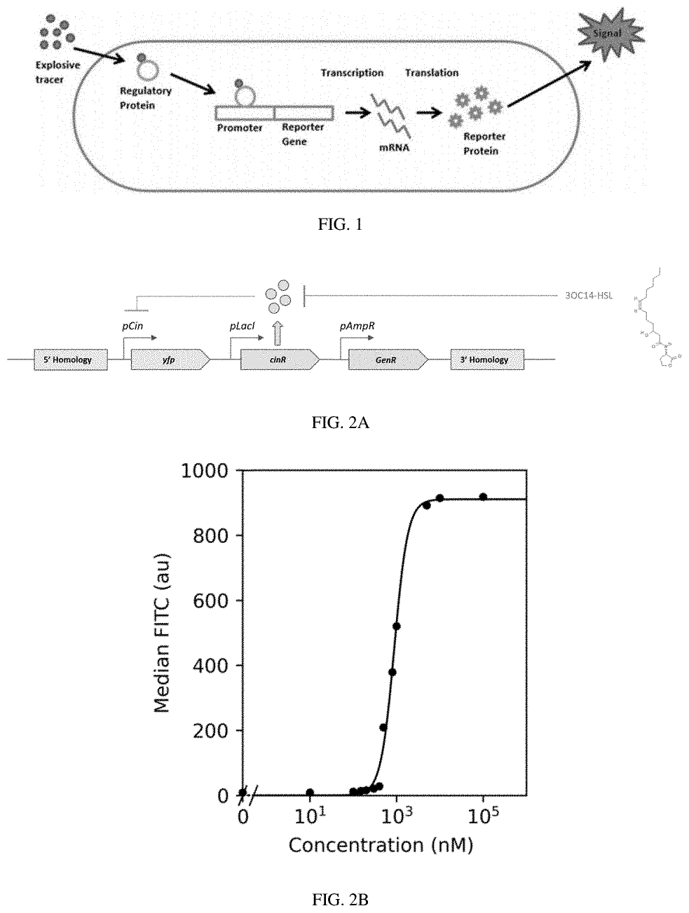

This example describes hyperspectral reporter systems used for the detection of either biologically- or non-biologically-derived targets of interest in field settings. In some embodiments, the hyperspectral reporter is produced by a living sensor featuring system components that detect a target of interest and in turn relay a measurable signal. In some embodiments, the living sensor is a microbe featuring an inducible regulatory protein that is activated by the microbe contacting a target of interest. In some embodiments, the target of interest can be an explosive tracer, a radioactive material, human DNA species, or a pathogenic DNA species. Upon induction of the regulatory protein by the target of interest, the regulatory protein triggers events leading to the synthesis of a proteinaceous gene product(s) that is capable of producing a measurable signal with hyperspectral photochemical properties (see FIG. 1).

An example of a biosensor capable of producing a hyperspectral reporter is set forth in FIG. 2. One example of a biosensor that was produced was an engineered Rubrivivax gelatinosus strain that comprising a genetic construct that is responsive to C14-homoserine lactone (HSL). The said example biosensor comprised a yellow fluorescent protein (YFP) YFP gene serving as a readout for the responsive of the biosensor to HSL. The presence of HSL inhibited the activity of a repressor protein which regulates the expression of YFP (see FIG. 2A). Flow cytometry analyses of the living sensors were incubated in varying amounts of HSL were used to construct a response curve which indicated that the living sensors were responsive to nanomolar concentrations of HSL (see FIG. 2B).

### Example 2

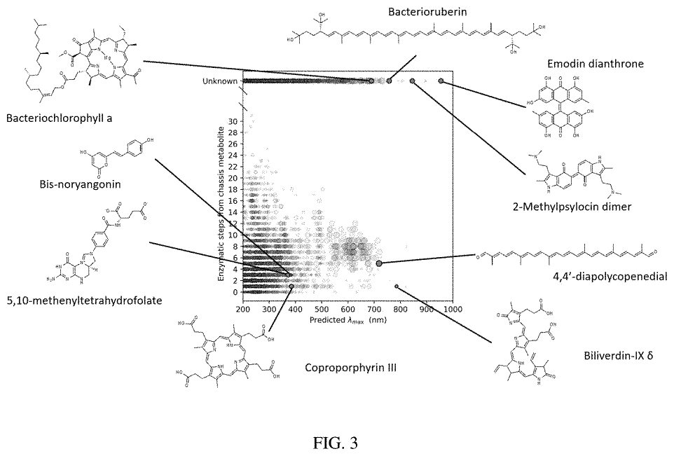

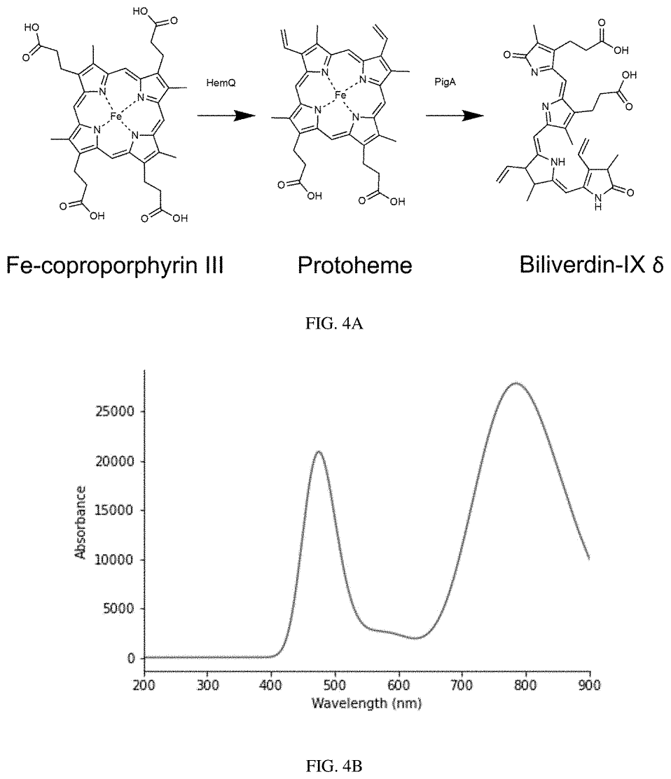

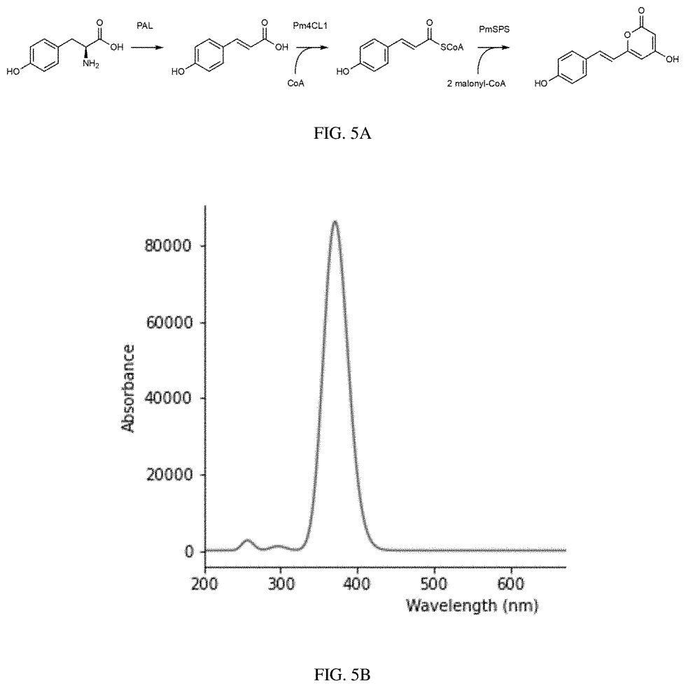

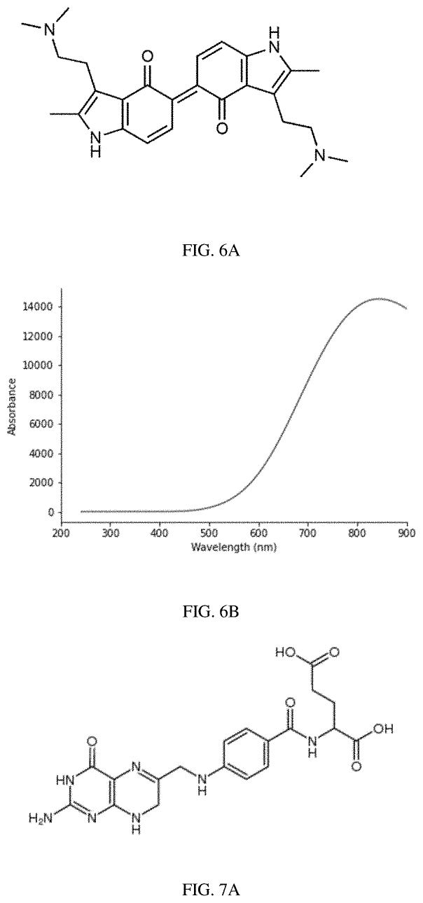

This example describes a computational approach that predicted molecules that may be able to function as hyperspectral reporter. Time-Dependent Density Function Theory (TDDFT) calculations were performed on 14,000 metabolites to predict their optical absorbance properties. Then, a network was generated from the reaction in the BKMS database and identified which metabolites are natively produced in E. coli and in B. subtilis, respectively. Then, a network search was performed to determine the minimal number of enzymes (from any kingdom) needed to access the metabolites not natively produced in E. coli or B. subtilis (see FIG. 3). Optical properties of promising candidate compounds were then predicted including properties associated with biliverdin-IX δ (see FIGS. 4A-4B), bis-noryangonin (see FIGS. 5A-5B), and bacteriochlorophyll a (see FIGS. 6A-6B).

### Example 3

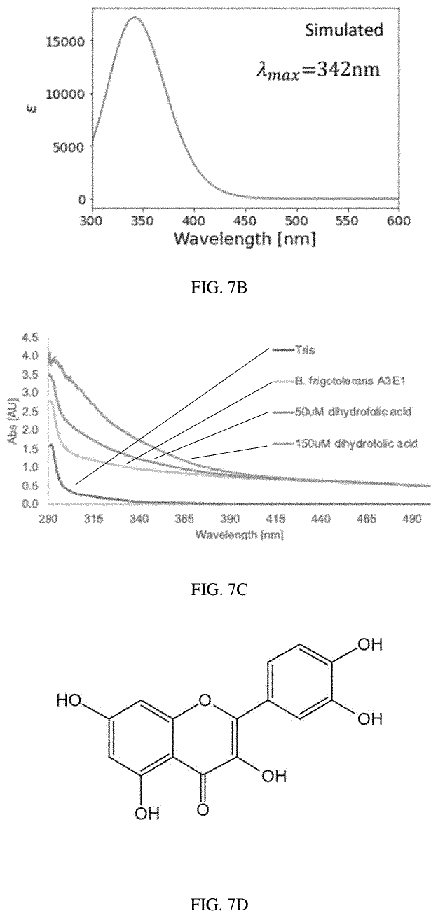

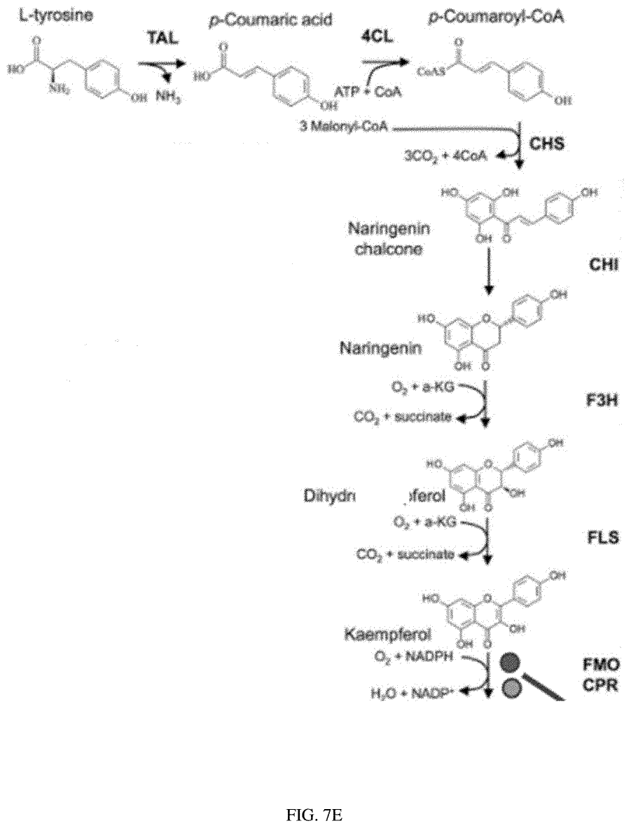

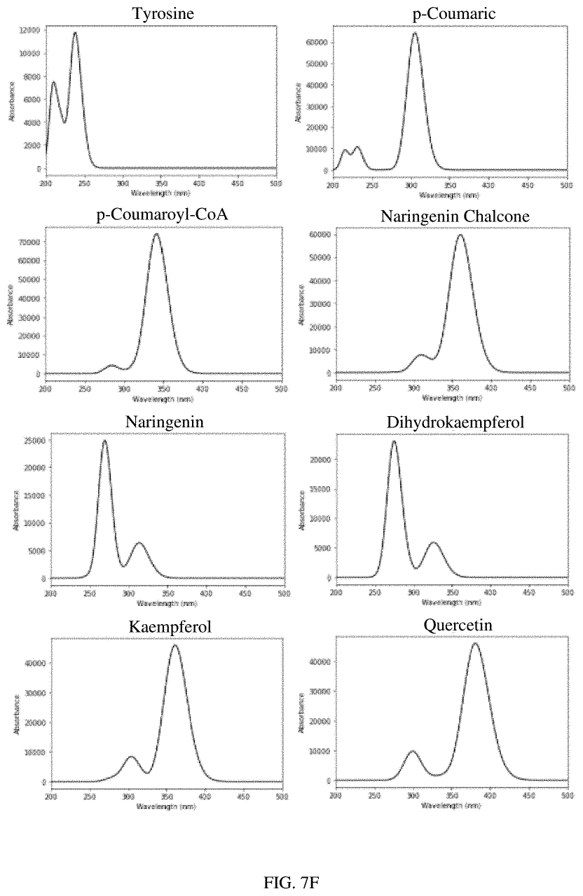

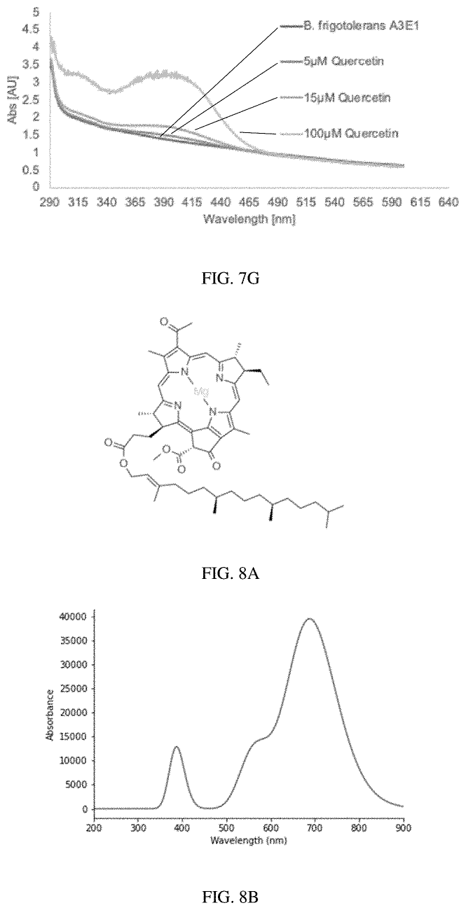

This example describes analysis of the photochemical properties of near-UV hyperspectral compounds dihydrofolic acid (see FIG. 7A) and quercetin (see FIG. 7D). TDDFT was used to predict the spectral signatures of both dihydrofolic acid (see FIG. 7B) and quercetin (see FIG. 7E). TDDFT simulations predicted that the absorption maxima of dihydrofolic acid and quercetin were 342 nm (see FIG. 7B) and 370 nm, respectively (see FIG. 7E). To determine the experimental photochemical properties of these hyperspectral compounds, varying concentrations of both dihydrofolic acid and quercetin solutions were subjected to absorption analyses across a range of selected wavelengths ranging from 290 nm-600 nm and compared to control samples of tris buffer and/or Brevibacterium frigoritolerans AE31 suspended in water (see FIG. 7B and 7E). Consistent with TDDFT simulations which predicted an absorption maxima for dihydrofolic acid of 342 nm (see FIG. 7B), solutions of 50 μM or 150 μM dihydrofolic acid both exhibited higher absorption values between wavelengths 290 nm-390 nm compared to control samples. Separately, 5 μM, 15 μM, and 100 μM solutions of quercetin all exhibited higher absorption values between wavelengths 315-465 nm compared to control samples (see FIG. 7F) which were also consistent with TDDFT simulations predicting an absorption maxima for quercetin at 370 nm (see 7 FIG. 2E). These results suggested that both dihydrofolic acid and quercetin exhibited absorption properties providing for detection using near-UV wavelengths of light.

### Example 4

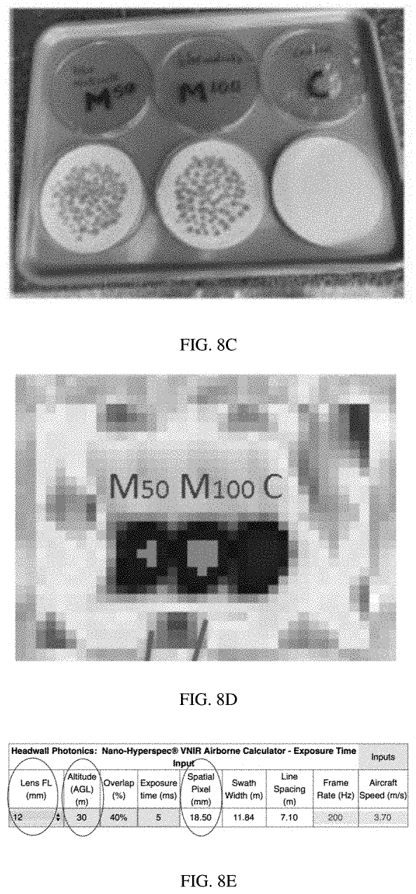

This example describes analysis of the photochemical properties of the near-infrared hyperspectral reporter bacteriochlorophyll a (see FIG. 8A). The molar absorption properties of bacteriochlorophyll a were predicted (see FIG. 8B). To confirm the utility of the hyperspectral properties of bacteriochlorophyll a, 10 cm plates were loaded with either no reporter or varying densities of purified bacteriochlorophyll a (see FIG. 8C). Then, the plates were imaged using a remote drone-mounted hyperspectral imaging device at an altitude of 30 m, featuring a lens focal length (FL) of 12 mm, employing an exposure time of 5 ms, and a frame rate of 200 Hz to detect the presence of bacteriochlorophyll a on the experimental plates (see FIGS. 8D-8E). At a spatial pixel resolution of 18.5 mm (see FIG. 8E), the remote drone-mounted hyperspectral imaging device detected clear signatures of bacteriochlorophyll a loaded on plates compared to control plates which exhibit near background levels of near-infrared signals (see FIG. 8D). This data indicated that drone-based imaging can be employed toward remote detection of near-infrared signatures of bacteriochlorophyll-harboring reporters.

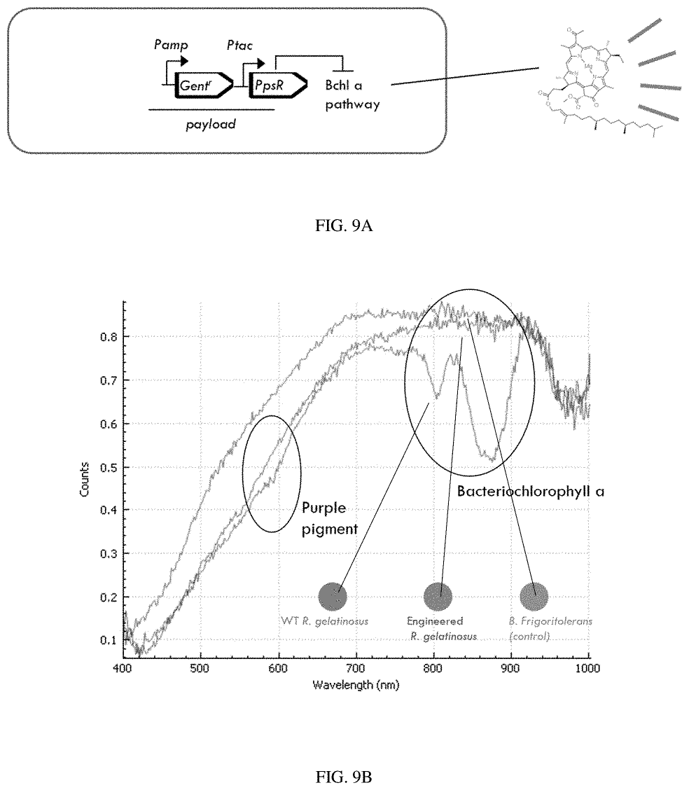

Genetic engineering of R. gelatinosus was employed to generate a strain capable of synthesizing bacteriochlorophyll a that could be further engineered to be used as a living sensor. Engineering of R. gelatinosus involved switching the promoter of PpsR photosynthesis master regulator to a strong but repressible promoter (see FIG. 9A). The hyperspectral phenotype of the engineered R. gelatinosus strain was then analyzed. When the hyperspectral spectrum of wild-type and B. frigoritolerans control bacteria were compared to the engineered R. gelatinosus strain, the results indicated that the genetic engineering process resulted in control of pigment and bacteriochlorophyll a production (see FIG. 9B).

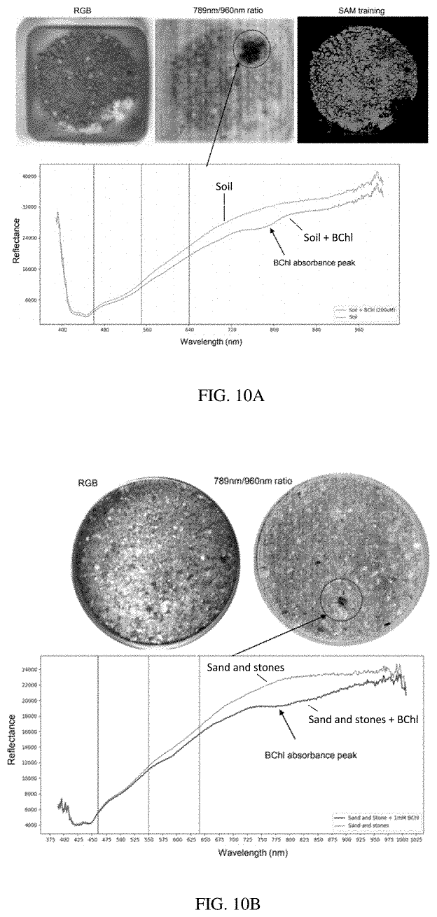

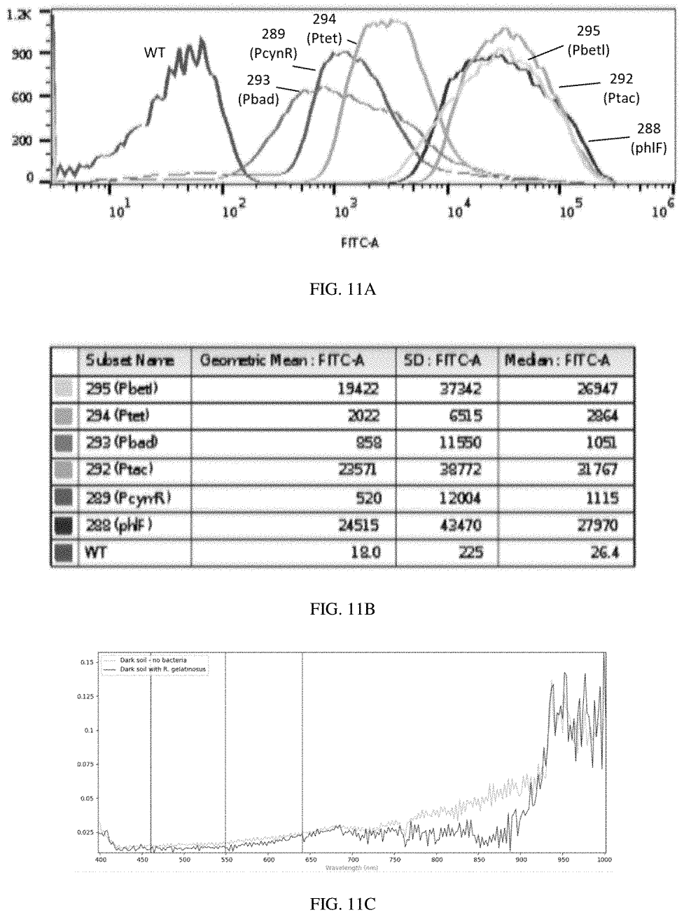

Apart from detection of bacteriochlorophyll near-infrared signatures on laboratory plates, bacteriochlorophyll a hyperspectral properties were also distinguishable in complex soil samples. To assess the detectability of bacteriochlorophylls in soil, solutions of bacteriochlorophyll a were seeded in either surface soil (see FIG. 10A) or mixed sand (see FIG. 10B) and then imaged through a variety of imaging approaches. While using an imaging filter that selects for visible light (such as red, green, and blue (RGB)) provided no detection of a 200 μM solution of bacteriochlorophyll a in surface soil, hyperspectral imaging filters which selects for light wavelength ratios of 789 nm/960 nm or spectral angle mapping provided robust detection of bacteriochlorophyll a which coincided with an absorption peak at ˜790 nm (see FIG. 10A). Similarly, while imaging a mixed sand sample seeded with 1 mM bacteriochlorophyll a using an RGB filter provided no detection of near-infrared signature, imaging filters which selects for light wavelength ratios of 789 nm/960 nm enabled visual detection of the presence bacteriochlorophyll a in mixed sand coinciding with an absorbance peak at ˜790 nm (see FIG. 10B).

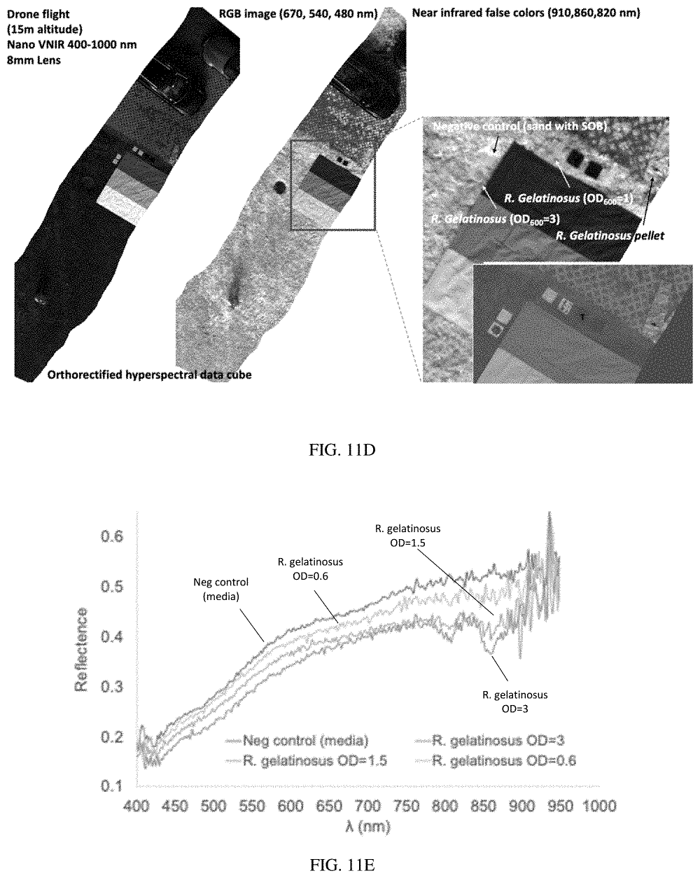

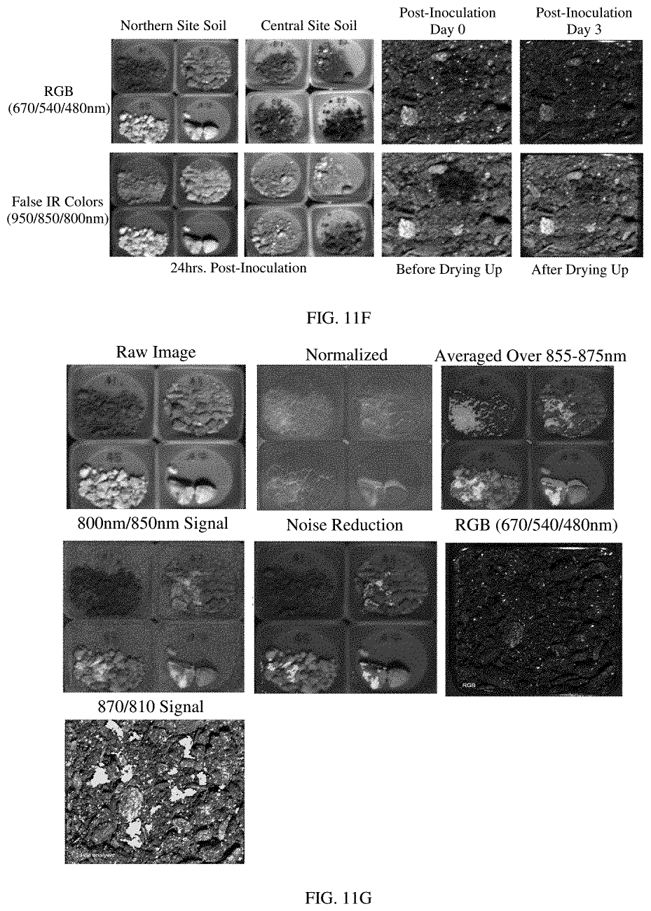

In addition to detection of purified bacteriochlorophyll a in seeded in complex soils, bacteria engineered to express bacteriochlorophyll a served as a hyperspectral compound when inoculated into complex soils. Flow cytometry analysis of bacteria provided a method for assessing promoter strength in order to enhance the detection limits of the hyperspectral detectable signal emitted from bacteriochlorophyll a (see FIGS. 11A-11B). When R. gelatinosus strains were engineered to express bacteriochlorophyll a the hyperspectral signal was detectible in soils (see FIGS. 11C-11D) in a concentration-dependent manner (see FIG. 11E). Moreover, R. gelatinosus seeded into soil were detectable up to 3 days post-inoculation and appeared as a pink-red hue in soil samples when analyzed with hyperspectral imaging devices (see FIG. 11F). Computational analyses of hyperspectral imaging data of R. gelatinosus strains expressing bacteriochlorophyll a highlighted increased light reflectance thereby enhancing the detection of the reporter cells in complex soils (see FIG. 11G). These results provided evidence that bacteriochlorophyll reporters provides hyperspectral imaging capabilities for detection of near-infrared signatures that are distinguishable even when the reporter signals are in low concentrations and present in complex soils as would be found in the field.

### Example 5

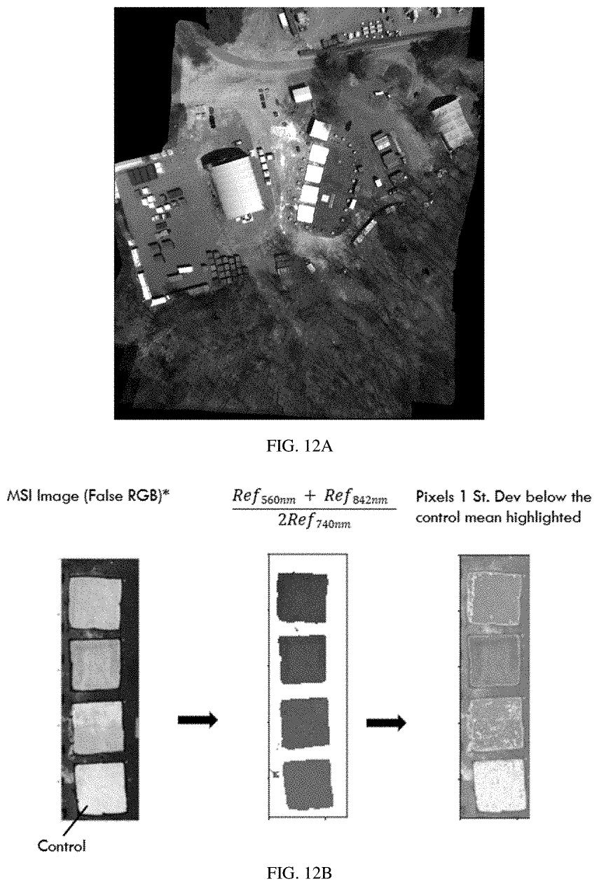

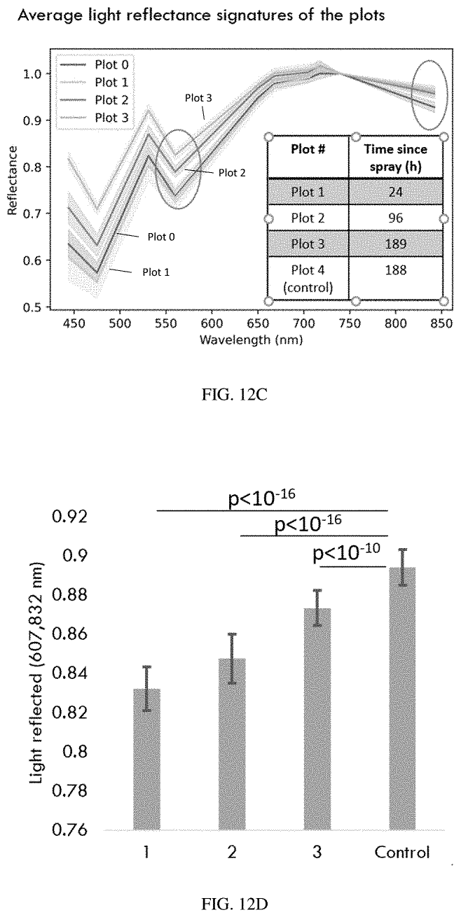

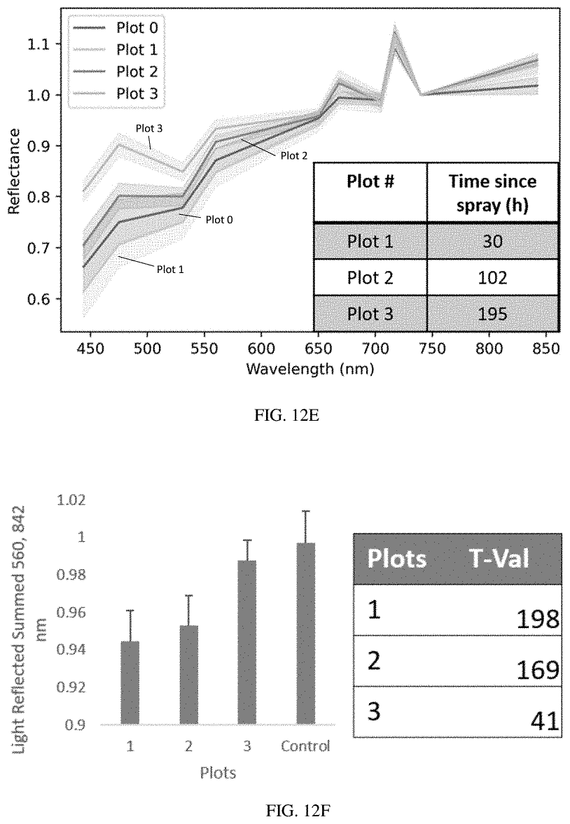

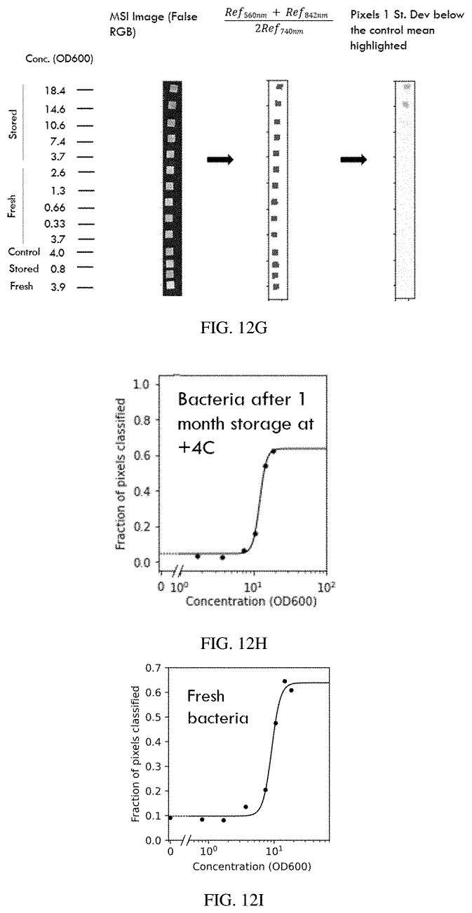

This example describes long-term classification of hyperspectral reporters. Respective plots were inoculated with hyperspectral reporters comprising R. gelatinosus comprising hyperspectral reporters at three different time points (such as 24, 96, and 189 hrs prior imaging) (see FIG. 12A). Then, the plots were imaged with a remote imaging device at various altitudes (such as 4.5, 9, 15, 30, 60, and 120 m). The images were then analyzed and the pixel intensities were quantified relative to R. gelatinosus that does not produce a signal (see FIGS. 12B-12F). These results indicated that hyperspectral signals were detectable in engineered R. gelatinosus that were not freshly deposited into the field. Analyses were then performed to determine a limit of detection of engineered R. gelatinosus. Plates were seeded with varying concentrations of fresh engineered R. gelatinosus and engineered R. gelatinosus stored at 4° C. for 1 month (see FIG. 12G). The results revealed that the detection limited of engineered R. gelatinosus did not change based on storage conditions (see FIGS. 12H-121).

### Example 6

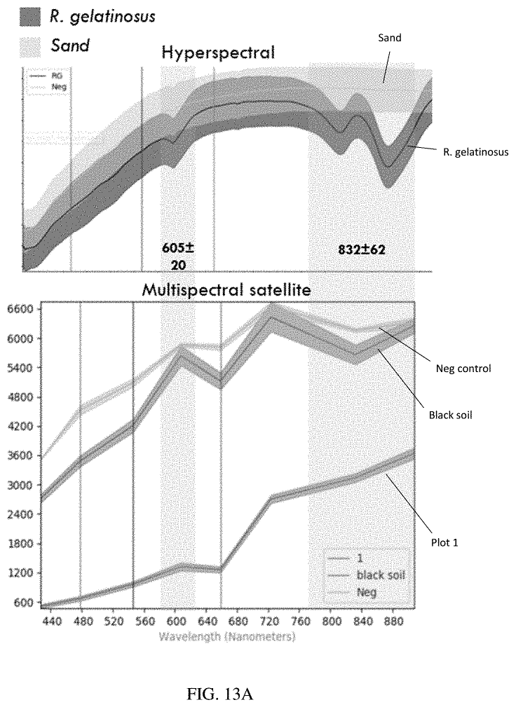

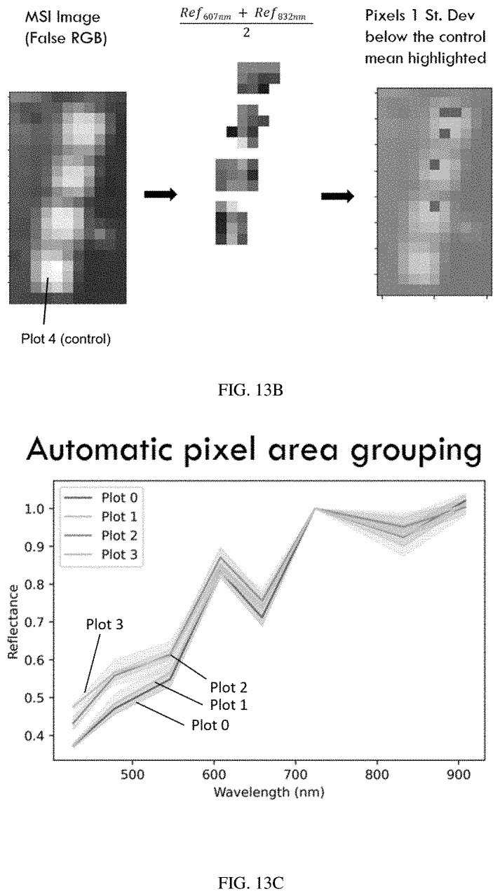

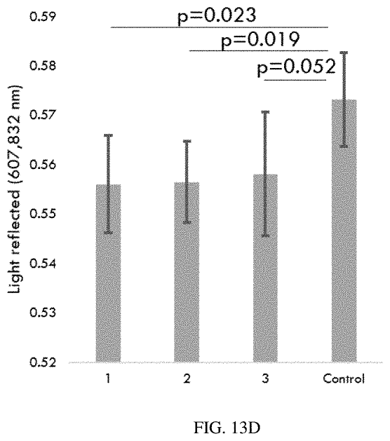

This example describes an experiment involving acquiring satellite images to measure the signal from an experiment site. Engineered R. gelatinosus was seeded at the experiment sites at 54, 136, and 218 hrs prior to imaging. The satellite images were acquired under the following conditions: orbit altitude was 770 km; orbit type was sun-synchronous; orbit period was 100 minutes; and revisit time was 1.1 days. The engineered R. gelatinosus at the experiment site was detected using yellow and NIR bands. The pixel size was 1.9×2.4 m² with each plot rendering 3-6 clean pixels per plot. The pixels were then normalized to the 724 nm and to control for differential light scattering caused by topography and other effects (see FIGS. 13A-13D). The results indicated that all three samples of engineered R. gelatinosus at the experiment site were detectable via satellite imaging.

### EMBODIMENTS

The following embodiments form aspects of the invention.

1. A plant cell, comprising

a transducer comprising a DNA molecule having a regulatory element responsive to a target input operatively linked to a DNA sequence encoding a signaling protein, wherein the transducer produces a detectable signal in response to the target input by producing the signaling protein, wherein the detectable signal features a unique hyperspectral signature.

2. The plant cell of embodiment 1, wherein the transducer is constitutively active in an engineered cell.

3. The plant cell of embodiment 1, wherein the transducer is purified from an engineered cell prior to exposing the transducer to a target.

4. The plant cell of embodiment 1, wherein the detectable signal is secreted extracellularly.

5. The plant cell of embodiment 1, wherein the detectable signal is not secreted extracellularly.

6. A hyperspectral reporter comprising,

an engineered cell comprising a transducer comprising a DNA molecule having a constitutively active regulatory element operatively linked to a DNA sequence encoding a signaling protein, wherein the transducer produces a detectable signal by producing the signaling protein, wherein the detectable signal features a unique hyperspectral signature.

7. The reporter of embodiment 6, wherein the engineered cell is a plant cell.

8. The reporter of embodiment 6, wherein the engineered cell is a bacteria.

9. The reporter of embodiment 6, wherein the signaling protein comprises the unique hyperspectral signature.

10. The reporter of embodiment 6, wherein the signaling protein is an enzyme and wherein the enzyme catalyzes the production of a metabolite comprising the unique hyperspectral signature from a substrate.

11. The reporter of embodiment 10, wherein the substrate is a naturally occurring compound in the cell.

12. The reporter of embodiment 6, wherein the signaling protein can trigger one or more steps in a biological pathway leading to the production of a compound comprising the unique hyperspectral signature.

13. The reporter of embodiment 6, wherein the detectable signal is produced by the unique hyperspectral signature of a compound selected from the group consisting of dihydrofolic acid, quercetin, bacteriochlorophyll a, bacteriochlorophyll b, oscillaxanthin, astaxanthin, beta-carotene, protoporphyrin IX naringenin-chalcone, kaempferol.

14. The reporter of embodiment 6, wherein the detectable signal is produced by the unique hyperspectral signature of a compound selected from the group consisting of E. coli metabolites, volatile metabolites, diterpenoids, phycocyanobilin.

15. The reporter of embodiment 6, wherein the detectable signal is produced by the unique hyperspectral signature of a compound having greater than or equal to 6, 7, 8, 9, 10, 11, 12, 13, 14, 15, or 16 conjugated π-bonds.

16. The reporter of embodiment 6, wherein the detectable signal absorbs light at a wavelength of 290 nm-460 nm or 790-810 nm.

17. The reporter of embodiment 6, wherein the target input is selected from a toxin, radiation, a pollutant, a nucleic acid, an explosive, and a nutrient.

18. The reporter of embodiment 17, wherein detectable signal represents a decreased level of an environmental nutrient, wherein the level is decreased with respect to a normal threshold level.

19. The reporter of embodiment 6, wherein the cell comprises 1-50 transducers, each transducer comprising a unique DNA molecule.

20. The reporter of embodiment 6, wherein the cell comprises 1-20 transducers, each transducer comprising a unique DNA molecule.

21. The reporter of embodiment 6, wherein the cell comprises 1-12 transducers, each transducer comprising a unique DNA molecule.

22. A method for detecting the presence of a live cell comprising detecting a signal having a unique hyperspectral signature, wherein the signal is generated from a hyperspectral reporter that comprises a living cell, wherein the signal is detected at least 10 meters from the living cell, wherein the signal provides information about the presence of the living cell.

23. The method of embodiment 22, wherein the hyperspectral signal is an invisible signal.

24. The method of embodiment 23, wherein the invisible signal is only detectable with a camera, such as HSI camera.

25. The method of embodiment 24, wherein a transgenic plant is remotely tracked.

26. The method of embodiment 24, wherein an engineered bacteria is remotely tracked.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A hyperspectral reporter comprising,
an engineered cell comprising a transducer comprising a DNA molecule having a regulatory element responsive to a target input operatively linked to a DNA sequence encoding a signaling protein, wherein the transducer produces a detectable signal in response to the target input by producing the signaling protein, wherein the detectable signal features a unique hyperspectral signature.

2. The hyperspectral reporter of claim 1, wherein the engineered cell is a bacterium, plant cell, or fungi, optionally wherein the bacterium is Rubrivivax gelatinosus.

3.-4. (canceled)

5. The hyperspectral reporter of claim 1, wherein the regulatory element is an inducible promoter.

6. The hyperspectral reporter of claim 1, wherein the signaling protein comprises the unique hyperspectral signature or the signaling protein can trigger one or more steps in a biological pathway leading to the production of a compound comprising the unique hyperspectral signature.

7. The hyperspectral reporter of claim 1, wherein the signaling protein is an enzyme and wherein the enzyme catalyzes the production of a metabolite comprising the unique hyperspectral signature from a substrate, optionally wherein the metabolite is produced from a substrate that is a naturally occurring compound in the cell.

8.-9. (canceled)

10. The hyperspectral reporter of claim 1, wherein the cell expresses a receptor capable of recognizing the target input, optionally wherein the cell comprises an exogenous nucleic acid encoding the receptor, optionally wherein the receptor is a peptidoglycan receptor.

11.-12. (canceled)

13. The hyperspectral reporter of claim 1, wherein the detectable signal is produced by the unique hyperspectral signature of a compound selected from the group consisting of dihydrofolic acid, quercetin, bacteriochlorophyll a, bacteriochlorophyll b, oscillaxanthin, astaxanthin, beta-carotene, protoporphyrin IX naringenin-chalcone, kaempferol, biliverdin-IX δ, bis-noryangonin, 2-methylpsylocin dimer, 5,10-methenyltetrahydrofolate, coproporphyrin III, bacterioruberin, emodin dianthrone, E. coli metabolites, volatile metabolites, diterpenoids, phycocyanobilin, or 4,4′-diapolycopenedial, wherein the compound has greater than or equal to 6, 7, 8, 9, 10, 11, 12, 13, 14, 15, or 16 conjugated π-bonds, or wherein the detectable signal absorbs light at a wavelength of 290 nm-460 nm or 790-810 nm, optionally wherein the detectable signal represents a decreased level of an environmental nutrient, wherein the level is decreased with respect to a normal threshold level.

14.-16. (canceled)

17. The hyperspectral reporter of claim 1, wherein the target input is selected from the group consisting of a toxin, radiation, a pollutant, a nucleic acid, an explosive, a bacterium, a fungus, a virus, and a nutrient.

18. (canceled)

19. The hyperspectral reporter of claim 1, wherein the cell comprises 1-50 transducers, wherein each transducer comprises a unique DNA molecule.

20.-21. (canceled)

22. A method for detecting a target input comprising
detecting a signal having a unique hyperspectral signature, wherein the signal is generated from a hyperspectral reporter that comprises a living cell, wherein the signal provides information about the detection of the target input.

23. The method of claim 22, wherein the living cell of the hyperspectral reporter is an engineered cell comprising a transducer comprising a DNA molecule having a regulatory element responsive to the target input operatively linked to a DNA sequence encoding a signaling protein, wherein the transducer produces the detectable signal in response to the target input by producing the signaling protein, wherein the signaling protein is an enzyme which catalyzes the production of a metabolite comprising the unique hyperspectral signature from a substrate, optionally wherein the substrate is a naturally occurring protein in the cell, the signaling protein comprises the unique hyperspectral signature, or the signaling protein can trigger one or more steps in a biological pathway leading to the production of a compound comprising the unique hyperspectral signature.

24. The method of claim 22, wherein the signal is detected remotely, at least about 10 meters away from the living cell, wherein the signal is detected using a hyperspectral camera, optionally wherein the hyperspectral camera is mounted on a drone airplane, or satellite.

25.-27. (canceled)

28. The method of claim 22, wherein the living cell is a bacterium or fungi, optionally wherein the living cell is Rubrivivax gelatinosus.

29.-33. (canceled)

34. The method of claim 22, wherein the target input is selected from the group consisting of a toxin, radiation, a pollutant, a nucleic acid, an explosive, a bacterium, a fungus, a virus, and a nutrient, optionally wherein the target input is present on crop seeds or is derived from soil, optionally wherein the nucleic acid is a pathogen nucleic acid or a human nucleic acid.

35.-37. (canceled)

38. The method of claim 34, wherein the target input is a nutrient and wherein the detectable signal is generated when the nutrient is present in a decreased level, wherein the level is decreased with respect to a normal threshold level or wherein the detectable signal is generated when the nutrient is present at a level within a normal range for that nutrient in that environment.

39.-41. (canceled)

42. The method of claim 22, wherein at least 2-50 detectable signals are generated from the living cell.

43.-48. (canceled)

49. The transducer method of claim 22, wherein the transducer is constitutively active in an engineered cell or wherein the transducer is purified from an engineered cell prior to exposing the transducer to a target.

50. (canceled)

51. The method of claim 22, wherein the detectable signal is secreted extracellularly or wherein the detectable signal is not secreted extracellularly.

52. (canceled)

53. A hyperspectral reporter comprising,
an engineered cell comprising a transducer comprising a DNA molecule having a constitutively active regulatory element operatively linked to a DNA sequence encoding a signaling protein, wherein the transducer produces a detectable signal by producing the signaling protein, wherein the detectable signal features a unique hyperspectral signature.

54. (canceled)

55. A hyperspectral reporter comprising, an engineered cell comprising a transducer comprising a DNA molecule having a regulatory element responsive to a target input operatively linked to a DNA sequence encoding a signaling protein, wherein the transducer produces a detectable signal in response to the target input by producing the signaling protein, wherein the detectable signal features a unique hyperspectral signature, wherein the target input also produces a physiological response.

56. The hyperspectral reporter of claim 55, wherein the physiological response is induced by at least one enzyme that is expressed as the result of the presence of the target input, wherein the physiological response produces additional substrates and/or proteins that are used to synthesize the detectable signal.

57.-59. (canceled)

60. The hyperspectral reporter of claim 55, wherein the physiological response makes the reporter responsive to a second target input, optionally wherein the presence of a second target input leads to the production of a second detectable signal featuring a unique hyperspectral signal and/or wherein the physiological response reduces the levels of the initial target input, optionally wherein the reduction of the initial target input is caused by the production of a biocontrol agent by the living cell.

61.-63 (canceled)
